(19) United States
(12) Patent Application Publication
(10) Pub. No.: US 2006/0068387 A1
(43) Pub. Date: Mar. 30, 2006

(54) Genetic marker for endocrine disorders

(75) Inventor: Andrea E Dunaif, Chicago, IL (US)

Correspondence Address: David A Casimir;Melden & Carroll, Suite 350, 101 Howard Street, San Francisco, CA, 94105, US

(73) Assignee: Northwestern University, Evanston, IL (US)

(21) Appl. No.: 10/511,710

(22) PCT Filed: Apr. 22, 2003
(86) PCT No.: PCT/US03/12820

§ 371 Date: Nov. 2, 2005

Related U.S. Application Data
(60) Provisional application No. 60/374,404, filed on Apr. 22, 2002.

(51) Int. Cl. C12Q 1/68

(52) U.S. Cl. 435/6

(57) ABSTRACT

A genetic marker associated with polycystic ovary syndrome is disclosed. The presence or absence of the allele is highly predictive of whether an individual is at risk from polycystic ovary syndrome. Methods of diagnosis, markers, and primers are disclosed and claimed in accordance with the present invention.

This application claims priority to U.S. Provisional Application No.: 60/374,404, filed Apr. 22, 2002. The aforementioned application is specifically incorporated herein by reference in its entirety.

This invention was supported in part with NIH grants U54 HD34449 and P50 HD44405. The United States government may have rights in this invention.

### FIELD OF THE INVENTION

The present invention relates to novel genetic markers for endocrine disorders. In addition, the present invention provides a genetic marker for the endocrine disorder polycystic ovary syndrome. In addition, methods of endocrine disorder diagnosis, markers, and primers are disclosed.

### BACKGROUND OF THE INVENTION

Polycystic ovary syndrome (PCOS) is a common endocrine disorder in premenopausal women, affecting 7-10% of this population. PCOS is an abnormality of the hypothalamic-pituitary-ovarian system. The major features of PCOS include menstrual dysfunction, anovulation, and signs of hyperandrogenism. The exact etiology is not clear. A characteristic of the syndrome is inappropriate gonadotropin secretion, which may be a result of, rather than a cause of, ovarian dysfunction. LH is tonically elevated throughout the menstrual cycle, Follicle Stimulating Hormone (FSH) is normal or low, the LH/FSH ratio is often greater than 3, and there is an exaggerated response of LH to gonadotropin-releasing hormone (GnRH).

Androgens such as testosterone, bioavailable testosterone, androstenedione and dehydroepiandrosterone sulfate (DHEAS), are frequently measurably elevated in the peripheral circulation, and these hormones and their metabolites account for the physical characteristics of the syndrome. The source of androgens may be from the ovaries, adrenals, or both.

In the early phase of the menstrual cycle, estradiol levels in women with PCOS are equal to those of normal women; however, mid-cycle elevations of estrogen and progesterone that normally occur after ovulation are absent. Because of the lack of cyclical progesterone secretion, the action of estradiol on both the hypothalamic-pituitary axis and the endometrium is unopposed. Both progesterone deficiency and acyclic estrogen production contribute to increased secretion of LH. The effects of unopposed estrogen on the endometrium may cause it to become hyperplastic, which may cause intermittent and heavy uterine bleeding and increase the long-term risk of endometrial cancer. These effects may be compounded, especially in obese patients, by increased levels of estrone converted from androstenedione in adipose tissue.

PCOS confers a substantially increased risk for impaired glucose tolerance (IGT) and type 2 diabetes mellitus (DM2), with prevalence rates of glucose intolerance approaching ˜40%. Women with PCOS have profound insulin resistance as well as pancreatic β-cell dysfunction, independent of obesity and glucose intolerance. However, skeletal muscle insulin resistance reverses in cultured myotubes suggesting that insulin resistance in this tissue is induced by factors in the in vivo environment. In addition, hyperandrogenemia is the reproductive phenotype in males as well as female relatives of PCOS women. Male relatives are also at risk for insulin resistance and type 2 diabetes. It is clear that PCOS-related insulin resistance is a risk factor for diabetes in the relatives of women with PCOS.

The classic reproductive symptoms of PCOS do not have their onset until after puberty. What is needed are genetic markers that can identify women at risk for PCOS and for diabetes associated with it. Genetic markers could also identify other relatives, such as brothers, who are at risk for PCOS-related diabetes. In addition, what is needed is a delineation of the specific mechanism of the metabolic phenotype associated with such genetic markers. What is also needed is a diagnostic test to identify subjects at risk for PCOS and to identify and test treatments of PCOS. What is further needed is an understanding of the pathogenesis of PCOS.

### SUMMARY

The present invention provides a genetic marker associated with polycystic ovary syndrome and related conditions. The presence or absence of the allele is highly predictive of whether an individual is at risk from polycysitc ovary syndrome and related conditions. Methods of diagnosis, markers, and primers are disclosed and accordance with the present invention.

The present invention is contemplated for use in the treatment of PCOS and related disorders (e.g., diabetes mellitus, diabetes insipidus, menstrual disorders, oligomenorrhea, amenorrhea, infertility, recurrent pregnancy losses, hirsutism, obesity, acne vulgaris, and other endocrine disorders).

It is contemplated that the present invention may be used within a health care setting in the treatment of PCOS and related disorders. For example, the present invention may be used as a pre-onset indicator of a person's likelihood of obtaining PCOS or a related disorder. In such a circumstance, the present invention may be used in the prediction of necessary lifestyle changes or medical interventions (e.g. monitoring, therapy, etc.) so as to reduce the likelihood of obtaining PCOS or a related disorder (e.g., dietary changes; exercise changes; etc) or to reduce or alleviate the severity or symptoms of PCOS or related disorders.

In preferred embodiments, the present invention provides a method to determine the presence or absence of polycystic ovary syndrome (PCOS) in an individual. In some embodiments, this method provides nucleic acid from an individual which is assessed for the presence or absence of a PCOS-associated allele 8+ (hereinafter A8(+)). In further embodiments, an absence of the allele in an individual indicates a likely absence of a PCOS causative gene in the genome of the individual and the presence of the allele a likely presence of the PCOS causative gene in the genome of the individual. In other embodiments, the assessing step is performed by a process that comprises subjecting the nucleic acid to amplification using oligonucleotide primers flanking at least a portion of D19S884. In still further embodiments, this method also involves the step oftreating an individual to prevent or ameliorate PCOS based on the results of the diagnostic method.

The present invention also provides a kit for the detection of the presence or absence of a PCOS-associated allele of D19S884. In further embodiemts, the kit provides reagents for detecting the allele and instructions for correlating the presence or absence of the allele to a medical intervention.

### DEFINITIONS

To facilitate an understanding of the invention, a number of terms are defmed below.

As used herein, the term “polycystic ovary syndrome” or “PCOS” when used in reference to alleles, genes, proteins, or chromosomal locations refers to markers correlated with polycystic ovary syndrome. The term “PCOS markers” encompasses both proteins and genes that are identical to wild-type PCOS and those that correlate to PCOS (e.g., through genetic linkage or through biological pathways).

As used herein, the term “instructions for using said kit for said detecting the presence or absence of a PCOS marker nucleic acid or polypeptide in said biological sample” includes instructions for using the reagents contained in the kit for the detection of PCOS markers. In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 51 0(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the MRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription andlor correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (ie., a sequence of nucleotides) related by the base-pairing rules. For example, for the sequence 5′-“A-G-T-3′,” is complementary to the sequence 3′-“T-C-A-5′.” Complementarity maybe “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (ie., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (ie., it is the complement of) the single-stranded nucleic acid sequence under conditions of low stringency as described above.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., Idnetics or thermodynamics) of binding by the first polypeptide may be the same as or greater than or less than the efficiency substrate binding by the second polypeptide. For example, the equilibrium binding constant (KD) for binding to the substrate may be different for the two polypeptides. The term “Km” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less. “High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μl/ml denatured salmon sperm DNA followed by washing in a solution comprising 0.1×SSPE, 1.0% SDS at 42 C. when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 gl/l NaCl, 6.9 g/l NaH₂PO₄ H₂O and 1.85 g/l EDTA, pH adjusted to 7.4 with NaOH), 0.5% SDS, 5× Denhardt's reagent and 100 μl/ml denatured salmon sperm DNA followed by washing in a solution comprising 1.0×SSPE, 1.0% SDS at 42 C. when a probe of about 500 nucleotides in length is employed.

“Low stringency conditions” comprise conditions equivalent to binding or hybridization at 42 C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5× SSPE, 0.1% SDS at 42 C. when a probe of about 500 nucleotides in length is employed. The present invention is not limited to the hybridization of probes of about 500 nucleotides in length. The present invention contemplates the use of probes between approximately 10 nucleotides up to several thousand (e.g., at least 5000) nucleotides in length.

One skilled in the relevant understands that stringency conditions may be altered for probes of other sizes (See e.g., Anderson and Young, Quantitative Filter Hybridization, in Nucleic Acid Hybridization [1985] and Sambrook et al., Molecular Cloning: A Laboratory Manual, Cold Spring Harbor Press, NY [1989]).

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each comprise a sequence (i.e., a portion of the complete polynucleotide sequence) that is similar between the two polynucleotides, and may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

As applied to polypeptides, the term “substantial identity” means that two peptide sequences, when optimally aligned, such as by the programs GAP or BESTFT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

The term “polymorphic locus” is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the term “genetic variation information” or “genetic variant information” refers to the presence or absence of one or more variant nucleic acid sequences (e.g., polymorphism or mutations) in a given allele of a particular gene or loci.

As used herein, the term “detection assay” refers to an assay for detecting the presence of absence of nucleic acid sequences (e.g., in a given allele or a particular gene). Examples of suitable detection assays include, but are not limited to, those described below in Section VI B.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-β replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those.to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (ie., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., MRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific MRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein, a “portion of a chromosome” refers to a discrete section of the chromosome. Chromosomes are divided into sites or sections by cytogeneticists as follows: the short (relative to the centromere) arm of a chromosome is termed the “p” arm; the long arm is termed the “q” arm. Each arm is then divided into 2 regions termed region 1 and region 2 (region 1 is closest to the centromere). Each region is further divided into bands. The bands may be further divided into sub-bands. For example, the 11p15.5 portion of human chromosome 11 is the portion located on chromosome 11 on the short arm (p) in the first region in the 5th band (5) in sub-band 5 (0.5). A portion of a chromosome may be “altered;” for instance the entire portion may be absent due to a deletion or may be rearranged (e.g., inversions, translocations, expanded or contracted due to changes in repeat regions). In the case of a deletion, an attempt to hybridize (i.e., specifically bind) a probe homologous to a particular portion of a chromosome could result in a negative result (i.e., the probe could not bind to the sample containing genetic material suspected of containing the missing portion of the chromosome). Thus, hybridization of a probe homologous to a particular portion of a chromosome may be used to detect alterations in a portion of a chromosome.

The term “sequences associated with a chromosome” means preparations of chromosomes (e.g., spreads of metaphase chromosomes), nucleic acid extracted from a sample containing chromosomal DNA (e.g., preparations of genomic DNA); the RNA that is produced by transcription of genes located on a chromosome (e.g., hnRNA and mRNA), and cDNA copies of the RNA transcribed from the DNA located on a chromosome. Sequences associated with a chromosome may be detected by numerous techniques including probing of Southern and Northern blots and in situ hybridization to RNA, DNA, or metaphase chromosomes with probes containing sequences homologous to the nucleic acids in the above listed preparations.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a MRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “antigenic determinant” as used herein refers to that portion of an antigen that makes contact with a particular antibody (ie., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein refers to a foreign, heterologous, or autologous gene that is placed into an organism by introducing the gene into newly fertilized eggs or early embryos. The term “foreign gene” refers to any nucleic acid (e.g. gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector”as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of MRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced PCOS transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention

The term “sample” as used herein is used in its broadest sense. A sample suspected of containing a human chromosome or sequences associated with a human chromosome may comprise a cell, chromosomes isolated from a cell (e.g. a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response,” when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat. Nos., 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

### DESCRIPTION OF THE FIGURES

FIG. 1 depicts a distribution of uT levels, in which a uT level of 15 ng/dL is 2 SD above the control mean and a value>15ng/dL was used to diagnose hyperandrogenemia.

FIG. 2 depicts a TDT analysis of chromosome 19p, D19S884 χ²=12.95, P=3.21×10⁻⁴ with 220 transmissions.

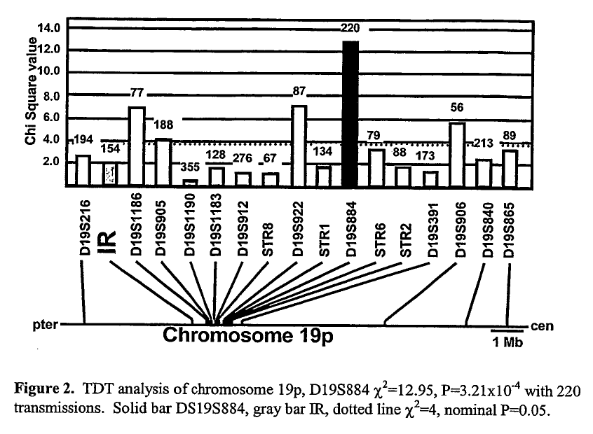

FIG. 3 depicts FSIGT glucose (A) and insulin (B) responses and 2 h post-75 g glucose (C) and insulin (D) levels in obese allele (A) A8(+) and A8(−) PCOS women. Tolbutamide, 500 mg iv, given at 20 min of the FSIGT. The shaded area in panel B is the difference in insulin responses in A8(+) vs A8(−) PCOS. *P<0.05 vs weight matched control women, **P<0.05 vs A8(−) PCOS, by ANCOVA adjusted for age.

FIG. 4 depicts fasting proinsulin, proinsulin:insulin and total triglyceride levels in obese A8(−) and A8(+) brothers of PCOS women, *P<0.05.

FIG. 5 depicts preferred embodiments for genetic variation resulting in androgen excess, which causes metabolic and reproductive defects by prenatal programming.

### GENERAL DESCRIPTION

Androgen excess or increased GnRH release can reproduce the PCOS reproductive phenotype. In addition, extreme insulin resistance secondary to mutations in the insulin receptor gene can cause the PCOS reproductive phenotype. Familial clustering of PCOS provides evidence for a genetic susceptibility to the disorder. PCOS is likely a complex genetic disease with at least several major susceptibility genes. It has been shown that the intermediate reproductive phenotype of hyperandrogenemia aggregates in PCOS families. Moreover, PCOS first-degree relatives with this reproductive phenotype also exhibit evidence of insulin resistance. Thus, identifying genes associated with the reproductive abnormalities is contemplated to also identify genes contributing to insulin resistance and related conditions (e.g., obesity). In support of this hypothesis, an allele has been identified, during the development of the present invention, of a marker in the region of the insulin receptor, allele A8 (hereinafter A8) of D19S884, that is both linked and associated with the reproductive phenotype. The marker in the region of the insulin receptor, A8 of D19S884, was identified through a family based association test for the association analysis, the transmission disequilibrium test (TDT), which tests for association in the presence of linkage and controls for population stratification. This association was replicated in a second sample of families. The association of this marker with PCOS has been confirmed in an independent case-control study, and the marker allele is associated with intermediate metabolic phenotypes.

Since association is dependent on the presence of linkage disequilibrium, and linkage disequilibrium is maintained over relatively short genetic distances, the evidence for association in the TDT analysis shows that D19S884 is close to the PCOS susceptibility gene and can provide a diagnostic marker.

Associations of a phenotype with a marker locus rather than a gene have been demonstrated in studies of maturity onset diabetes of the young (MODY). Such studies defined these MODY metabolic phenotypes well before the gene linked to the marker locus was positionally cloned. The association between quantitative metabolic phenotypes and anonymous chromosomal markers has also been investigated in diabetes genome scans. Moreover, the presence of an association between the marker locus and a metabolic phenotype provides additional evidence for a susceptibility gene near the marker locus.

Only three candidate genes for PCOS have been identified in linkage studies: CYP11a (cholesterol side-chain cleavage enzyme), the insulin gene variable number of tandem repeats (VNIR) and follistatin. There has been linkage and association using family-based analyses with an allele of the insulin gene VNTR locus and insulin levels in PCOS. Further studies of follistatin and CYP11a have not supported a major role for variation in either of these genes in susceptibility to PCOS. Evidence suggests a lack of an association between the insulin VNTR and PCOS in family studies. Other putative candidate genes for PCOS have been identified in case-control studies. Polymorphisms in insulin receptor substrate (IRS)-1, IRS-2, PPAR-gamma pro12ala allele have been associated with metabolic phenotypes in a recent case-control studies. However, case-control studies must be interpreted with caution since they are particularly susceptible to false positive results due to population stratification. Therefore, a focus is needed on the D19S884 region identified by the present invention since A8 is both linked and associated with reproductive and metabolic phenotypes in PCOS. As additional genes or marker loci that meet these stringent criteria are discovered, investigation is needed in their association with the phenotypic features of PCOS.

There is profound peripheral insulin resistance in PCOS similar in magnitude to that seen in DM2. However, the mechanism of insulin resistance in PCOS differs from that seen in DM2 or obesity. It has been shown that serine-phosphorylation of the insulin receptor (IR) is caused by an extrinsic serine kinase and results in decreased IR signaling in cultured PCOS fibroblasts. The presence of a serine kinase inhibiting IR phosphorylation in PCOS fibroblasts has been confirmed recently in an independent laboratory. Post-IR signaling defects are selective, affecting metabolic but not mitogenic pathways in PCOS fibroblasts. There are post-IR signaling defects in PCOS skeletal muscle, and preliminary studies suggest that these also impair metabolic but not mitogenic pathways. Further, the pattern of changes in signaling proteins in skeletal muscle differs from that in other insulin resistant conditions such as DM2, obesity and gestational diabetes. PCOS skeletal muscle does not exhibit significant differences in the abundance of the IR, IRS-1, or the p85 regulatory subunit of phosphatidylinositol-3 (PI3)-kinase. The abundance of IRS-2 is increased, suggesting that this change is compensatory for decreased IRS-1 mediated signaling.

Skeletal muscle is the major target tissue on a quantitative basis for insulin-mediated glucose disposal (IMGD) in vivo, accounting for 85% of glucose utilization in the fed state. Based on studies in PCOS fibroblasts, it was hypothesized that insulin resistance was a genetic defect and that skeletal muscle would have persistent defects in insulin action as a stable phenotype in culture, similar to findings in DM2. To test this hypothesis, insulin action was examined in cultured myotubes from PCOS and control women. In contrast to cultured skeletal muscle from DM2, no evidence for intrinsic decreases in insulin sensitivity in PCOS cultured skeletal muscle was detected. However, PCOS cultured skeletal muscle was not entirely similar to control because there were significant increases in basal, non-insulin-mediated glucose uptake and constitutive activation of mitogen-activated protein kinase (MAPK) pathways. Activation of MAPK was also present in PCOS muscle biopsies indicating this finding was not an artifact of tissue culture conditions. These increases in MAPK activity are another unique feature of the PCOS insulin resistance phenotype and are not seen in DM2. These findings suggest that the primary defect in PCOS is not skeletal muscle insulin resistance.

The studies in fibroblasts and skeletal muscle indicate that there are tissue differences in insulin action; similar findings have been reported in mice with disruption of insulin signaling pathways. In contrast to skeletal muscle findings, McAllister has reported constitutive increases in p38 stress-activated MAPK and decreased p44MAPK in passaged PCOS theca cells. Ciaraldi and colleagues first proposed that such tissue differences in insulin action may account for continued insulin actions on the ovary in the face of resistance to insulin's metabolic actions. The selective nature of insulin resistance in PCOS with preservation and even enhancement of growth-related MAPK pathways may also contribute to reproductive actions of insulin in the face of resistance to its metabolic actions.

The observation of reversible skeletal muscle defects in insulin action has led to a re-formulated hypothesis for the pathogenesis of insulin resistance in PCOS. It is now proposed that a circulating factor causes insulin resistance in vivo in PCOS. Candidate factors include free fatty acids (FFA), cytokines such as tumor necrosis factor (TNF)-α or resistin, and androgens. There is an increasing body of evidence from human and animal studies to support the hypothesis that skeletal muscle insulin resistance in various settings is an acquired defect. Further, it is proposed that intrinsic alterations in skeletal muscle (e.g. activation of MAPK) increase susceptibility to the insulin-resistance inducing effects of the circulating factors, and it has been shown that cultured PCOS skeletal muscle have increased susceptibility to FFA-mediated insulin resistance.

Androgens represent a possible circulating factor that could produce acquired defects in insulin action in PCOS. Women with upper-body obesity share many features of PCOS, such as insulin resistance, increased subcutaneous abdominal adipocyte size and abnormalities in the regulation of lipolysis. Since women with upper-body obesity often have increased androgen production, it is possible that androgens are a common final path for these metabolic defects in PCOS and upper-body obesity. However, the hypothesis that androgens play a major role in the pathogenesis of insulin resistance in PCOS has been largely discounted because suppressing androgens does not normalize insulin action in PCOS. Further, suppressing androgens does not alter resistance to β-adrenergic receptor agonists in isolated adipocytes in PCOS.

It has been suggested that adiposity accounts for insulin resistance in PCOS. In Scandinavian PCOS women, insulin sensitivity could be completely normalized by weight reduction. However, abnormalities in insulin secretion persisted. In obese PCOS women matched to control women for visceral fat, no significant differences in insulin sensitivity or EGP existed. In contrast, it has been shown that lean PCOS women matched to control women for total fat mass and waist:hip ratios (WHR) had significantly decreased IMGD. However, increases in visceral adipose tissue (VAT) can escape detection with anthropometric measurements, so it remains possible that these lean PCOS women had increased VAT. Few studies have quantitated VAT in PCOS, and there are conflicting reports as to whether it is increased compared to control women matched for total fat mass. Lean PCOS women have increased abdominal fat cell size, a correlate of increased visceral adipose mass. A synergistic negative effect of adiposity and PCOS on EGP has also been found, suggesting that adiposity had a greater impact in PCOS than in reproductively-normal women. Holte and colleagues reported similar findings for insulin sensitivity. Increased VAT could, in turn, be a consequence of androgen programming.

The classic candidate mediators of adiposity-related insulin resistance are FFA. However, the importance of FFA in the pathogenesis of insulin resistance in women has been challenged recently. A study has demonstrated gender differences in susceptibility to FFA-mediated peripheral insulin resistance: men were susceptible to this FFA action whereas women were not. New evidence from cultured skeletal muscle supports the presence of such differences in susceptibility to FFA-mediated insulin resistance. PCOS skeletal muscle is more susceptible to this FFA action than cultured skeletal muscle from control women. This mechanism could account for the greater deleterious effect of adiposity on insulin action that has been observed in PCOS. Circulating FFA levels have not been well-studied in PCOS, nor are there studies of FFA flux in the disorder. A recent study found that lipolysis was increased in PCOS visceral fat. This difference could lead to increased portal FFA levels, which in turn could induce hepatic and peripheral insulin resistance. FFA levels could also be increased in PCOS because of decreased suppression of lipolysis due to the relative decreased insulin secretion that is also found in the syndrome. It remains possible that other fat-cell derived factors, adipokines, such as TNF-α, contribute to adiposity- related insulin resistance in PCOS.

The fetal origins or Barker hypothesis proposes that intrauterine growth retardation (IUGR), as evidenced by low birth weight, causes insulin resistance, cardiovascular disease and other features of the insulin resistance syndrome. Decreased fetal nutrition is proposed to result in decreased fetal insulin secretion and growth. Insulin resistance is a compensatory mechanism that further decreases fetal nutrient use: the “thrifty” phenotype. Extensive animal studies support the long-term impact of the fetal environment on the adult animal, known as fetal programming. The molecular mechanisms for these phenomena remain largely unknown, but permanent alterations in gene expression produced by changes in gene methylation may play a role. Many epidemiologic studies in humans support the association between low birth weight and metabolic diseases. Currently, the Barker hypothesis has not been tested prospectively in humans. However, the long-term consequences, such as obesity and glucose intolerance, of fetal hyperinsulinemia, which results in high birth weight in the offspring of diabetic mothers, have been documented by Boyd Metzger, and his colleagues. Thus, it is clear in humans that there are permanent physiologic alterations related to the intrauterine environment.

Sex steroids are well known to produce sex-specific differentiation in a number of fetal tissues, such as the urogenital tract and the brain. Less appreciated are the programming actions of androgens that alter metabolism. Transient exposure to androgens in several animal models can permanently decrease insulin sensitivity and secretion, as well as hepatic clearance of insulin. Androgens can also alter body fat distribution and lipolysis. Thus, androgen programming can recreate many features of the PCOS metabolic phenotype including insulin resistance, β-cell dysfunction, catecholamine resistance in subcutaneous abdominal adipocytes, but increased visceral adiposity and sensitivity to catecholamine-mediated lipolysis in this fat depot. Prenatally androgenized female rhesus monkeys are also smaller for gestational age. Indeed, prenatally androgenized monkeys have many of the reproductive features of PCOS: increased LH levels, irregular ovulation, polycystic ovaries and functional ovarian hyperandrogenism. Some of the androgen programming effects depend on the sex of the animal.

There is evidence for fetal origins of some features of PCOS in human studies. Ibanez and colleagues have reported that both girls with elevated adrenal androgen levels or with PCOS were significantly smaller for gestational age than reproductively normal control girls. The androgen programming could account for the recently reported sex differences in susceptibility to FFA-mediated insulin resistance and explain why women with PCOS appear to have the male phenotype for this effect.

While all of the above studies provide clues to the biology underlying PCOS, such studies do not provide methods for diagnosing PCOS. The present invention provides such methods by describing genetic markers that correlate to PCOS and related conditions.

### DETAILED DESCRIPTION

A genetic marker associated with endocrine disorders (e.g., polycystic ovary syndrome) is disclosed. The presence or absence of the polymorphic allele is highly predictive of whether an individual is at risk for polycystic ovary syndrome and related conditions. Methods of diagnosis, markers, and primers are disclosed.

Exemplary compositions and methods of the present invention are described in more detail in the following sections: I. Familial Aggregation of Hyperandrogenemia and Insulin Resistance in PCOS Families; II. Genetic Analyses; m. Genotype-Phenotype Analyses; IV. Correlation Between PCOS and Protein Expression and/or Activity; V. Mechanisms for Acquired Defects in Insulin in PCOS: Role of FFA and TNF-A in PCOS; and VI.Detection of PCOS Alleles.

I. Familial Aggregation of Hyperandrogenemia and Insulin Resistance in PCOS Families

In some embodiments, the present invention provides a genetic marker for endocrine disorders. In particular embodiments, the genetic marker is present for hyperandrogenemia in PCOS kindreds. In further embodiments, the genetic marker is present for hyperandrogenemia in PCOS kindreds with 46% of sisters affected. In other embodiments, approximately 50% of such sisters flfill diagnostic criteria for PCOS with chronic anovulation (≦6 menses/year) and hyperandrogenemia. In other embodiments, the other 50% of affected sisters present a novel phenotype: hyperandrogenemia (HA) with regular menses.

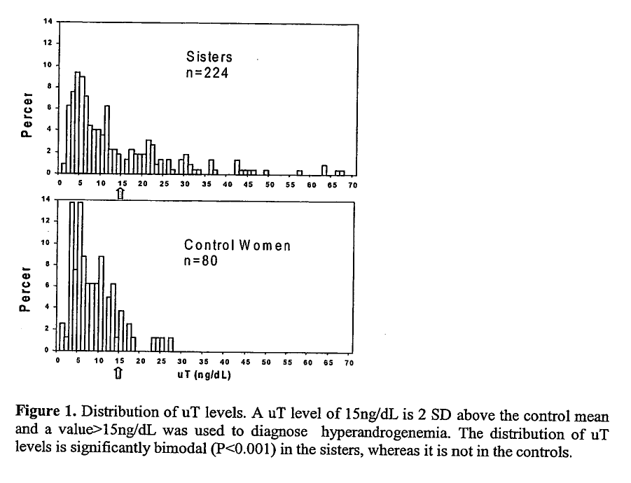

Sisters of women with the genetic marker provided by the present invention present a significant bimodal distribution of testosterone (T) levels, as opposed to a unimodal distribution in control women, as presented in FIG. 1. This bimodal distribution is consistent with a monogenic trait controlled by two alleles of an autosomal gene.

Familial aggregation of metabolic defects is present in first-degree relatives of individuals with the genetic marker provided by the present invention. There is familial aggregation of insulin resistance in PCOS consistent with the genetic marker provided by the present invention. Hyperandrogenemia and insulin resistance track together suggesting that they may reflect variation in the same gene or in closely linked genes.

Brothers of individuals with PCOS present unique phenotypes. Premature male balding in the brothers of individuals with PCOS was not detected. Brothers of individuals with PCOS have a reproductive phenotype with elevated DHEAS levels. Brothers of women with PCOS have significantly elevated DHEAS levels (e.g., 3035±1132 brothers vs 2492±1172 ng/mL control men, P<0.05). In addition, such DHEAS levels present a significant positive linear relationship between DHEAS levels in PCOS probands and their brothers (e.g., r=0.35, P=0.001). It is contemplated that elevated DHEAS levels in brothers of individuals with PCOS reflects an underlying abnormality in steroidogenesis similar to premenopausal sisters of PCOS women.

Fasting glucose levels do not differ in brothers of individuals with PCOS compared to controls. Fasting insulin and proinsulin levels in brothers of individuals with PCOS do not statistically differ in comparison with controls (e.g. insulin levels 16±9 brothers vs 14±8 μU/mL controls, P=0.07; proinsulin levels 15±12 vs 11±6 pmol/L, P=0.08). The proinsulin:insulin molar ratio, a marker of β-cell function, is not increased in brothers of individuals with PCOS. There are significant positive correlations between both insulin levels (e.g., r=0.27, P<0.05) and proinsulin levels (e.g., r=0.54, P<0.001) in brothers and their proband sisters with PCOS. Total TTG levels are significantly increased in PCOS brothers (e.g., 191±153 brothers vs 144±95 controls mg/dL, P<0.05). There is no significant difference between brothers of individuals with PCOS and controls regarding cholesterol, HDL or LDL levels. Brothers of PCOS women have insulin resistance and lipid abnormalities associated with the insulin resistance syndrome. It is contemplated that the phenotypic traits observed in families of individuals with PCOS are heritable traits and may be predicted by the compositions and methods of the present invention.

II. Genetic Analyses

An association exists between the genetic marker provided by the present invention and the insulin receptor (IR) and follistatin (FS). The combined phenotype of PCOS or hyperandrogenemia (PCOS/HA) with an affected sib pair (ASP) reveals linkage association with follistatin. The combined phenotype of PCOS/HA with an ASP reveals linkage association with markers in the region of the IR. In some embodiments, it is contemplated that FS and the IR are candidate genes for PCOS and are analyzed in the methods of the present invention.

In preferred embodiments, the present invention provides the genetic marker D19S884. In other preferred embodiments, the genetic marker D19S884 associates with PCOS. In further embodiments, the D19S884 marker links to and associates with the IR region. A TDT analysis indicates that D19S884 is in linkage disequilibrium with the PCOS susceptibility gene.

The marker D19S884 maps to 1 Mb centromeric to the IR. There are several known genes in this region: 1) SCYA25, a thymus-expressed cytokine, 2) MAP2K7, a mitogen activated serine/threonine kinase that activates c-Jun N-terminal kinase (JNK) in response to activation by growth factors, cytokines and stress and 3) resistin, a recently identified cytokine, which is expressed in adipocytes, down-regulated by thiazolidinediones, and that induces insulin resistance in rodents.

III. Genotype-Phenotype Analyses

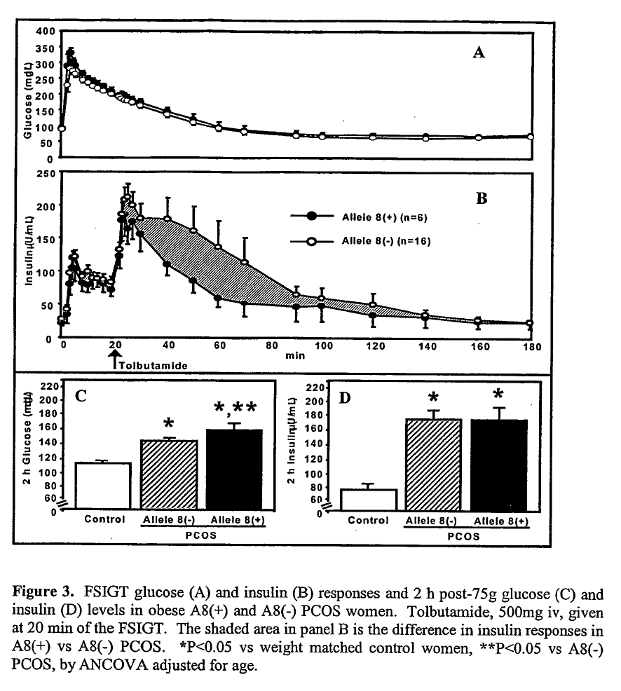

In preferred embodiments, the present invention provides the PCOS/HA allele - A8 of D1 9S884 (hereinafter A8(+)). In further embodiments, the A8+allele is associated with metabolic phenotypic features in PCOS women. Approximately 30% of PCOS women are A8(+). The frequency of allele 8 families is 20.6%. There are no significant differences in fasting glucose or insulin levels or in glucose:insulin ratios between A8(+) and A8(−) PCOS individuals. Similar degrees of insulin resistance exist in A8(+) and A8(−) PCOS women. However, 2 hour post-challenge glucose levels are significantly increased in A8(+) PCOS compared to A8(−) PCOS and to control women (P<0.05, ANCOVA), see Table 1 and FIG. 3. Post-challenge insulin levels do not differ in the A8(+) and A8(−) PCOS groups and are higher than in the control women. The adrenal androgen DHEAS is higher in A8(+) women. A8(+) and A8(−) PCOS women have virtually identical insulin sensitivity (SI) values indicating that they have a similar degree of insulin resistance. However, glucose levels are slightly higher and insulin levels substantially lower in A8(+) PCOS women (see FIG. 3). Insulin responses to tolbutamide are much lower in A8(+) PCOS women. As such, it is contemplated that A8(+) women possess a defect in the sulfonylurea receptor. A defect in the sulfonylurea receptor within A8(+) PCOS women may be an androgen-medicated action on ATP-sensitive potassium K⁺_{ATP} channels in the β-cell.

TABLE 1
Non-Hispanic White Obese PCOS (mean ± SEM)

| | A8(+) | A8(−) | |
|---|---|---|---|
| | (n) | (n) | P |
| AGE | 27 ± 1 | 30 ± 1 | 0.008 |
| yr | (82) | (160) | |
| BMI | 37.4 ± 0.8 | 38.4 ± 0.6 | 0.3 ^{a,b} |
| kg/m² | (82) | (160) | |
| Systolic | 126 ± 2 | 122 ± 1 | 0.06 ^{a,b} |
| mm/Hg | (60) | (119) | |
| Diastolic | 76 ± 1 | 75 ± 1 | 0.2 ^{a} |
| mm/Hg | (60) | (118) | |
| 0 h Glucose | 93 ± 2 | 91 ± 0 | 0.2 ^{a} |
| mg/dL | (81) | (160) | |
| 2 h Glucose | 153 ± 8 | 137 ± 4 | 0.03 ^{a} |
| mg/dL | (32) | (82) | |
| 0 h Insulin | 29 ± 2 | 29 ± 1 | 1.0 ^{a} |
| μU/mL | (81) | (158) | |
| 2 h Insulin | 168 ± 16 | 167 ± 14 | 0.5 ^{a} |
| μU/mL | (31) | (80) | |
| Proinsulin | 25 ± 2 | 22 ± 1 | 0.3 ^{a} |
| pmol/L | (77) | (152) | |
| FSIGT | | | |
| AGE | 26 ± 3 | 27 ± 1 | 0.9 |
| yr | (6) | (16) | |
| SI | 2.0 ± 0.5 | 2.0 ± 0.5 | 1.0 |
| x10⁻⁴/min/μU/mL | (6) | (16) | |
| SG | 2.1 ± 0.2 | 1.8 ± 0.1 | 0.3 |
| x10⁻² | (6) | (16) | |
| DI | 115 ± 23 | 126 ± 24 | 0.8 |
| AIRg | 57 ± 14 | 67 ± 5 | 0.4 |
| μU/mL | (6) | (16) | |
| AUC Insulin ₂₋₁₀ | 680 ± 105 | 806 ± 57 | 0.3 |
| μU/mL | (6) | (16) | |
| AUC Glucose ₀₋₁₈₀ | 19729 ± 1424 | 18222 ± 574 | 0.2 |
| mg/dL | (6) | (16) | |
| AUC Insulin ₀₋₁₈₀ | 10615 ± 2871 | 15254 ± 2869 | 0.2 |
| μU/mL | (6) | (16) | |
| AUC | 0.51 ± 0.10 | 0.81 ± 0.13 | 0.09 |
| Iusulin:Glucose ₀₋₁₈₀ | (6) | (16) | |

^{a} ANCOVA Adjusted for Age
^{b} Interaction

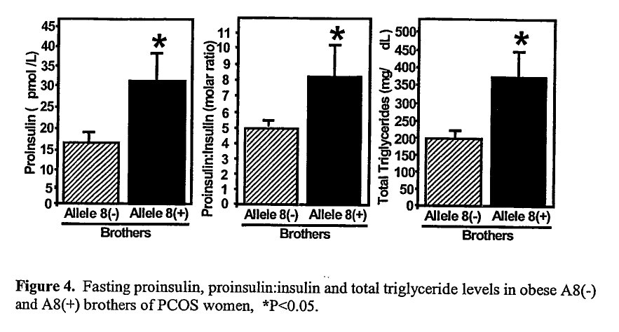

Phenotypic differences exist in A8(+)and A8(−) obese brothers of PCOS probands. Proinsulin levels, proinsulin:insulin molar ratios and TTG levels are significantly increased in A8(+) brothers compared to A8(−) brothers (see FIG. 4). HDL levels are lower in A8(+) brothers (e.g., 35±2 A8(+) vs 40±2 A8(−) mg/dL, P=0.053). In yet other embodiments, DHEAS levels are higher in A8(+) brothers.

A8 has no detected impact on metabolic parameters of normal individuals. A8(+) obese unaffected sisters present no significant differences in fasting metabolic parameters.

A8(+) PCOS women have significant changes in body mass index (BMI) with age and in post-challenge glucose levels. A8(+) PCOS women present a failure of compensatory insulin secretion in the A8(+) PCOS. A8(+) PCOS women also present decreased insulin secretion. The magnitude of insulin resistance is similar in the A8(+) and A8(−) PCOS women.

A8(+) brothers also appear to have a metabolic phenotype consistent with the insulin resistance syndrome. The increase in proinsulin levels and the proinsulin:insulin molar ratio in A8(+) brothers suggests that they may also have β-cell dysfunction. The present invention provides methods for detecting A8(+) brothers of PCOS women. In particular embodiments, proinsulin levels and proinsulin:insulin molar ratios are used to detect A8(+) brothers of PCOS women. In further embodiments, proinsulin levels are higher in A8(+) brothers, see FIG. 4, than in A8(+) PCOS probands, see Table 1. There are sex differences in the metabolic phenotype between A8(+) brothers and sisters of PCOS women.

IV. Correlation Between PCOS and Protein Expression and/or Activity

Table 2 shows differences in protein expression and/or activity between PCOS and control samples. There are no significant differences in the fold-stimulation of glucose transport or glucose incorporation into glycogen between PCOS myoblasts and control myoblasts (see Table 2). There are significant increases in basal glucose transport in PCOS myoblasts compared to control myoblasts. Increases in basal glucose transport in PCOS myoblasts compared to control myoblasts is due to increases in GLUTI abundance. GLUT4 abundance was similar in PCOS and control myotubes. Metabolic signaling pathways are similar in PCOS and control myotubes. However, mitogenic pathways are upregulated in PCOS myotubes in comparison with control myotubes. Basal mitogen-activated protein kinase kinase (MEK) phosphorylation is increased, and insulin-stimulated MEK phosphorylation is significantly increased in PCOS without a change in MEK abundance. p44/42 MAPK phosphorylation is significantly increased at baseline and in response to insulin in PCOS without any change in the abundance of these signaling proteins. There is a significant increase in p44/42 MAPK phosphorylation at baseline in PCOS skeletal muscle biopsies (e.g., 63±9 PCOS n=8 vs 30±6 n=8 control, % internal standard, P<0.05) without changes in MAPK abundance.

TABLE 2
PCOS and Control Cultured Myotubes (mean ± SEM)

| | Control | PCOS | |
|---|---|---|---|
| | (n = 8) | (n = 7) | P |
| Glucose transport | | | |
| Basal nmol/mg/min | 13.4 ± 1.3 | 19.8 ± 2.2 | 0.02 |
| 100 nM | 19.0 ± 2.1 | 26.8 ± 3.2 | 0.06 |
| Fold | 1.4 ± 0.4 | 1.35 ± 0.04 | NS |
| Basal nmol/mg/h | 4.7 ± 0.5 | 7.0 ± 1.2 | NS |
| 100 nM | 12.1 ± 1.5 | 19.4 ± 4.5 | NS |
| Fold | 2.6 ± 0.1 | 2.7 ± 0.2 | NS |
| Protein abundance | | | |
| IRβ* | 87 ± 38 | 93 ± 13 | NS |
| IRS-1* | 154 ± 30 | 253 ± 46 | 0.07 |
| IRS-2* | 69 ± 11 | 76 ± 15 | NS |
| p85* | 136 ± 52 | 118 ± 36 | NS |
| GLUT 1* | 106 ± 32 | 184 ± 48 | 0.02 |
| GLUT 4* | 96 ± 7 | 96 ± 8 | NS |
| Tyrosine | | | |
| phosphorylation | | | |
| IRβ 100 nM* | 34 ± 6 | 39 ± 4 | NS |
| IRS-1 100 nM* | 42 ± 15 | 32 ± 11 | NS |
| P13-kinase activity | | | |
| IRS-1 - associated | | | |
| Fold | 17 ± 2 | 18 ± 6 | NS |
| IRS-2 - associated | | | |
| | 8 ± 1 | 9 ± 1 | NS |
| Phospho MAPK | | | |
| p44/42* | | | |
| Basal | 28 ± 10 | 94 ± 9 | 0.003 |
| 100 nM | 68 ± 23 | 206 ± 35 | 0.02 |
| | 122 ± 22 | 108 ± 13 | NS |
| | 49 ± 45 | 88 ± 20 | NS |
| | 118 ± 45 | 293 ± 47 | NS |
| | 196 ± 32 | 157 ± 32 | 0.04 |

*% internal standard

There are constitutive increases in glucose uptake, GLUTI abundance and p44/42 MAPK activation in PCOS myotubes. Activation of growth related MAPK pathways has not been found in other insulin resistant states and is another unique feature of the PCOS phenotype. The p44/42 MAPK pathway is activated by growth factors such as insulin and regulates cell proliferation, cell survival and gene expression. Enhanced signaling through MAPK pathways, basally and in response to insulin, contributes the PCOS phenotype.

V. Mechanisms for Acquired Defects in Insulin Action in PCOS: Role of FFA and Tumor Necrosis Factor (TNF)-α in PCOS

Decreases in IMGD and IRS-1-associated P13-kinase activity in PCOS occuring in cultured skeletal muscle are acquired secondarily. Candidate factors that could modulate insulin sensitivity include androgens, FFA, TNF-α, resistin and adiponectin. Fasting FFA levels are significantly increased in obese PCOS compared to control women of comparable age and weight, despite higher fasting insulin levels in PCOS women—which is consistent with resistance of FFA suppression by insulin in vivo. TNF-α levels are not significantly increased in obese PCOS compared to weight-comparable control women.

There is increased sensitivity to FFA or TNF-α actions in PCOS women. Palmitate causes a significantly greater decrease in both basal as well as insulin-stimulated glycogen synthesis in PCOS than in control women, whereas TNF-α has a similar effect in decreasing glycogen synthesis in PCOS and control myotubes.

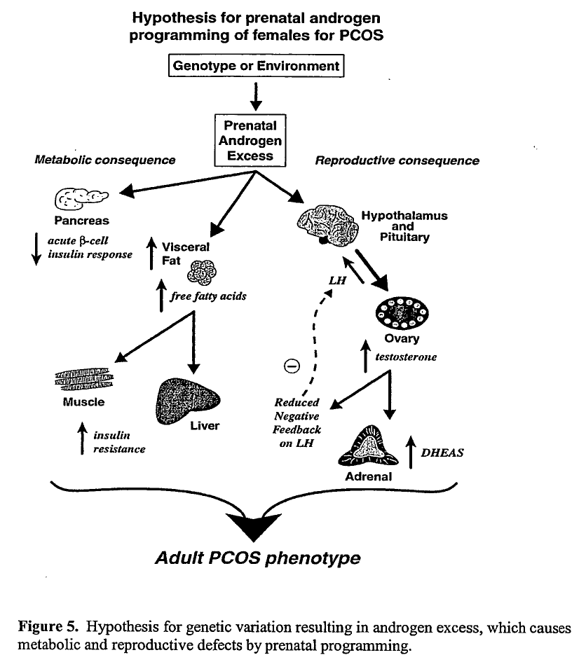

Hyperandrogenemia is caused by a variation in a gene regulating steroidogenesis. Androgen excess causes metabolic abnormalities in PCOS. Steroidogenic abnormality leads to increased androgen production by the fetal ovary and adrenal. Resulting intrauterine androgen excess results in increased LH release and decreased insulin secretion. Androgens alter LH release and insulin secretion by changing the activity of K⁺_{ATP} channels in GnRH neurons and pancreatic β-cells. Androgens also program adipose tissue resulting in increased visceral adiposity and increased sensitivity of these adipocytes to catecholamine-mediated lipolysis. Androgen related changes result in increased FFA delivery to the liver, which increases hepatic glucose production. Intrauterine androgen programming decreases hepatic clearance of insulin and alters muscle insulin action. A8 was identified during development of the present invention in linkage studies with the reproductive phenotype of hyperandrogenemia. In adults, however, there are no significant differences in androgen levels in A8(+) compared to A8(−) PCOS women. A8(+) results in prenatal androgen excess. Insulin sensitivity appears to be similar in A8(+) and A8(−) adult PCOS. The major effect of A8 is on insulin secretion. Additional genetic factors contribute to insulin resistance in PCOS since defects in insulin action are present in A8(−) PCOS. See FIG. 5 for a detailed analysis diagram.

VI. Detection of PCOS Related Alleles

In some embodiments, the present invention provides methods of detecting the presence of PCOS markers.

A. PCOS Alleles

In some embodiments, the present invention includes alleles of PCOS that increase a patient's susceptibility to PCOS (e.g., including, but not limited to, the genetic marker D19S884). However, the present invention is not limited to this particular marker. Any marker that correlates to D19S884 and PCOS finds use with the present invention.

B. Detection of PCOS Alleles

Accordingly, the present invention provides methods for determining whether a patient has an increased susceptibility to PCOS by determining whether the individual has a PCOS marker. In other embodiments, the present invention provides methods for providing a prognosis of increased risk for PCOS related symptoms (e.g. hyperandrogenemia) to an individual based on the presence or absence of PCOS markers.

A number of methods are available for analysis of nucleic acid sequences. Assays for detection of nucleic acid sequences fall into several categories, including, but not limited to direct sequencing assays, fragment length polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

1. Direct Sequencing Assays

In some embodiments of the present invention, sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the marker sequence) is sequenced using any suitable method, including but not limited to manual sequencing using radioactive marker nucleotides, or automated sequencing. The results of the sequencing are displayed using any suitable method. The sequence is examined and the presence or absence of a given marker sequence is determiined.

2. PCR Assay

In some embodiments of the present invention, variant sequences are detected using a PCR-based assay. In some embodiments, the PCR assay comprises the use of oligonucleotide primers that hybridize only to the allele to be detected.

3. Mutational Detection by dHPLC

In some embodiments of the present invention, sequences are detected using a PCR-based assay with consecutive detection of nucleotide variants by dHPLC (denaturing high performance liquid chromatography). Exemplary systems and methods for dHPLC include, but are not limited to, WAVE (Transgenomic, Inc; Omaha, Neb.) or VARIAN equipment (Palo Alto, Calif.).

4. Fragment Length Polymorphism Assays

In some embodiments of the present invention, sequences are detected using a fragment length polymorphism assay. In a fragment length polymorphism assay, a unique DNA banding pattern based on cleaving the DNA at a series of positions is generated using an enzyme. DNA fragments from a sample containing a marker sequence will have a different banding pattern than samples without the marker.

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from experimental and control samples.

5. Hybridization Assays

In preferred embodiments of the present invention, sequences are detected using a hybridization assay. In a hybridization assay, the presence of absence of a given marker or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest is detected directly by visualizing a bound probe (e.g., a Northern or Southern assay, See e.g., Ausabel et aL (eds.), Current Protocols in Molecular Biology, John Wiley & Sons, NY [1991]). In a these assays, genomic DNA (Southem) or RNA (Northem) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the sequence being detected is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, sequences are detected using a DNA chip hybridization assay. In this assay, a series of oligonucleotide probes are affixed to a solid support. The oligonucleotide probes are designed to be unique to a given marker. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given marker are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding,

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the marker of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given marker. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

6. Mass Spectroscopy Assay

In some embodiments, a MassARRAY system (Sequenom, San Diego, Calif.) is used to detect sequences (See e.g., U.S. Pat. Nos. 6,043,031; 5,777,324; and 5,605,798; each of which is herein incorporated by reference). DNA is isolated from blood samples using standard procedures. Next, specific DNA regions containing the marker of interest are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

7. Kits for Analyzing Risk of PCOS

The present invention also provides kits for determining whether an individual contains a PCOS marker. In some embodiments, the kits are useful determining whether the subject is at risk of developing PCOS. The diagnostic kits are produced in a variety of ways. In some embodiments, the kits contain at least one reagent for specifically detecting a PCOS marker. In preferred embodiments, the reagent is a nucleic acid that hybridizes to nucleic acids containing the marker and that does not bind to nucleic acids that do not contain the marker. In other preferred embodiments, the reagents are primers for amplifying the region of DNA containing the marker.

In some embodiments, the kit contains instructions for determining whether the subject is at risk for developing PCOS. In preferred embodiments, the instructions specify that risk for developing PCOS is determined by detecting the presence or absence of a PCOS marker in the subject, wherein subjects having a marker are at greater risk for PCOS.

The presence of absence of a disease-associated marker in a PCOS gene can be used to make therapeutic or other medical decisions.

In some embodiments, the kits include ancillary reagents such as buffering agents, nucleic acid stabilizing reagents, protein stabilizing reagents, and signal producing systems (e.g., florescence generating systems as FRET systems). The test kit may be packages in any suitable manner, typically with the elements in a single container or various containers as necessary along with a sheet of instructions for carrying out the test. In some embodiments, the kits also preferably include a positive control sample.

8. Bioinformatics

In some embodiments, the present invention provides methods of determining an individual's risk of developing PCOS based on the presence of PCOS markers. In some embodiments, the analysis of sequence data is processed by a computer using information stored on a computer (e.g., in a database). For example, in some embodiments, the present invention provides a bioinformatics research system comprising a plurality of computers running a multi-platform object oriented programming language (See e.g. U.S. Pat. No. 6,125,383; herein incorporated by reference). In some embodiments, one of the computers stores genetics data (e.g., the risk of contacting a particular disease or condition, as well as the sequences). In some embodiments, one of the computers stores application programs (e.g., for analyzing the results of detection assays). Results are then delivered to the user (e.g., via one of the computers or via the Internet).

For example, in some embodiments, a computer-based analysis program is used to translate the raw data generated by the detection assay (e.g., the presence, absence, or amount of a given PCOS marker) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence of PCOS marker), specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of developing PCOS) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### EXAMPLES

### Example 1

### Familial Aggregation of Hyperandrogenemia and Insulin Resistance in PCOS Families

The evidence to support the genetic analysis of a complex trait is familial aggregation. This finding was present for hyperandrogenemia in PCOS kindreds with 46% of sisters thus affected. Only one-half of these sisters fulfilled diagnostic criteria for PCOS with chronic anovulation (≦6 menses/year) and hyperandrogenemia. The remaining affected sisters had a novel phenotype: hyperandrogenemia (HA) with regular menses. There was a significant bimodal distribution of testosterone (T) levels in the sisters whereas the distribution was unimodal in the control women (see FIG. 1). The bimodal distribution was consistent with a monogenic trait controlled by two alleles of an autosomal gene . This study strongly suggested that hyperandrogenemia in PCOS had a genetic basis and that a possible candidate gene would be one involved in the regulation of both ovarian and adrenal steroidogenesis since levels of the adrenal androgen dehydroepiandrosterone sulfate (DHEAS) were also increased. Next, it was determined whether familial aggregation of metabolic defects was present in first-degree relatives. To control for the confounding effects of ethnicity on insulin sensitivity, the population to Non-Hispanic White women was limited. Two hundred seventeen sisters of 165 PCOS probands and 47 ethnically-comparable, reproductively-normal control women were studied. Phenotypes were defined as PCOS: ≦6 menses/yr and an elevated total or biologically available (u) (T) level; HA: menses every 27-35 d and an elevated T or uT level; Unaffected (UA): menses every 27-35 d and normal T, uT, and DHEAS levels (see Table 3). It was concluded that there was familial aggregation of insulin resistance in PCOS consistent with a genetic trait. Hyperandrogenemia and insulin resistance tracked together suggesting that they may reflect variation in the same gene or in closely linked genes.

TABLE 3
Metabolic Parameters in Sisters of PCOS Probands (mean ± SD)

| | PCOS | HA | UA | Control | P |
|---|---|---|---|---|---|
| Glucose | 89 ± 9^{c} | 83 ± 8 | 87 ± 8^{c} | 84 ± 8 | <0.001 |
| mg/dL | | | | | |
| Insulin | 24 ± 11^{a} | 19 ± 12^{a} | 14 ± 7 | 14 ± 8 | <0.001 |
| μU/mL | | | | | |
| Glucose:Insulin | 4.6 ± 2.3^{a} | 5.6 ± 2.6^{a} | 73 ± 2.6^{b} | 7.5 ± 3.5 | <0.001 |
| Ratio | | | | | |

^{a}significant vs UA and control;
^{b}significant vs control;
^{c}significant vs HA and control;
^{d}significant vs HA and UA;
^{e}significant vs HA, UA, and control

Next, it was determined whether a male phenotype was present in the brothers of PCOS women. One hundred nineteen brothers of 87 unrelated women with PCOS and 68 weight- and ethnicity-comparable unrelated control men were studied. Premature male balding in the brothers (a suggested male phenotype in previous studies) was not detected. Brothers of women with PCOS had significantly elevated DHEAS (3035±1132 brothers vs 2492±1172 ng/mL control men, P<0.05). There was a significant positive linear relationship between DHEAS levels in PCOS probands and their brothers (r=0.35, P=0.001). It was concluded that the PCOS brothers appeared to have a reproductive phenotype with elevated DHEAS levels. The elevated DHEAS levels might reflect the same underlying abnormality in steroidogenesis for which evidence has been found in ˜50% of the premenopausal sisters of PCOS women.

Fasting glucose levels did not differ in brothers compared to controls. Both fasting insulin (16±9 brothers vs 14±8 μU/mL controls, P=0.07) and proinsulin (15±12 vs 11±6 pmol/L, P=0.08) levels tended to be higher in the PCOS brothers, but this difference did not achieve statistical significance in this sample. The proinsulin:insulin molar ratio, a marker of β-cell function, was not increased in the brothers. There were significant positive correlations between both insulin levels (r=0.27, P<0.05) and proinsulin levels (r=0.54, P<0.001) in brothers and their proband sisters with PCOS, suggesting that these were also heritable traits in PCOS families. Total TTG levels were significantly increased in PCOS brothers (191±153 brothers vs 144±95 controls mg/dL, P<0.05). There were no significant differences in cholesterol, HDL or LDL levels. These findings also suggested that the brothers of PCOS women had insulin resistance and lipid abnormalities associated with the insulin resistance syndrome.

### Example 2

### Genetic Analyses

It was next determined whether there was linkage between polymorphic markers at candidate genes and the combined phenotype of PCOS or hyperandrogenemia (PCOS/HA) with an affected sib pair (ASP) analysis . Association in the presence of linkage with the TDT analysis in PCOS proband-parent trios was tested. 37 candidate genes (33 chromosomal locations) were screened involved in steroidogenesis, gonadotropin secretion, insulin action, or energy metabolism in 168 families and 39 affected sib pairs (ASP). Significant evidence for linkage with follistatin was found (identity by descent [IBD]=72%, χ²=1 2.97, nominal P=3.2 ×10⁻⁴, P<0.01 corrected for multiple tests) and for association with markers in the region of the IR by the TDT. However, the association findings in the region of the IR were not significant after a very stringent correction for multiple testing at the time of that publication. It was concluded that follistatin (FS) and the IR were high priority candidate genes for PCOS.

The possibility of genetic variation in FS was next investigated. Such variation might lead to overexpression or increased binding activity of FS that could contribute to the pathogenesis of PCOS by resulting in arrested folliculogenesis, increased thecal androgen secretion, decreased FSH release, and decreased insulin secretion. No evidence was found for sequence variants that play a major role in PCOS. A nominally significant association of a single nucleotide polymorphism in exon 6 by the TDT analysis was found that did not remain significant after correction for multiple tests. There were no differences in FS expression in PCOS fibroblasts. It was concluded that variation in FS did not play a major role in PCOS.

In the original series the marker D19S884 in the region of the IR showed the strongest evidence for association in 168 trios in the TDT analysis, χ²=8.53, although this result was not significant after using a Bonferroni correction for testing ˜350 alleles . However, in the second data set of 190 trios, D19S884 still has the strongest evidence for association, χ²=8.84, as well as in the combined data set of 358 trios, χ²=12.95, P=3.21×10⁻⁴. There is now also evidence for linkage in the IR region with IBD=63%, χ²=8.784, P=3.04×10⁻³ in 98 ASP. In addition, a case-control study also found that an allele ofDl 9S884 was significantly associated with PCOS, providing support of the findings in an independent sample. This association finding has been replicated in a second sample of families as well as in an independent case-control study, and there is also evidence for linkage using the ASP analysis. The evidence for association in the TDT analysis suggests that D19S884 is in linkage disequilibrium with the PCOS susceptibility gene. Taken together, these findings provide strong evidence to implicate a gene close to D19S884 in susceptibility to PCOS. Moreover, since the required sample to detect “signal” is inversely related to the increase in disease susceptibility conferred by a gene, these significant findings in a fairly small sample size suggest that this locus contains a major susceptibility gene for PCOS.

### Example 3

### Genotype-Phenotype Analyses

An investigation focused on whether the PCOS/HA allele, A8 of D19S884, was associated with any metabolic phenotypic features in PCOS women. To control for the confounding effect of obesity and ethnicity on insulin action, the population was limited to obese Non-Hispanic White individuals. Homozygous and heterozygous carriers of D19S884 A8 in the A8(+) group were identified. Approximately 30% of PCOS women are A8(+) by this definition. The frequency of allele 8 Centre d−Etude du Polymorphisme Humain (CEPH) families was 20.6%. Control women were age-, ethnicity-, and BMI-comparable, reproductively-normal women (n=64). Data on 75 g OGTT 0 h and 2 h glucose and insulin responses in 32 A8(+) PCOS and 81 A8(−) PCOS was obtained, as shown in Table 2. FSIGT studies in 6 A8(+) PCOS and 16 A8(−) PCOS were next performed. In the OGTT group, A8(+) PCOS women were significantly younger than the A8(−) group so all analyses were adjusted for age by analysis of covariance (ANCOVA). There was a significant age*BMI interaction (P<0.05), and BMI increased with age in A8(+) and remained stable in A8(−). A similar trend was found for blood pressure but did not achieve statistical significance (P=0.06). There were no significant differences in fasting glucose or insulin levels or in glucose:insulin ratios. These parameters were significantly increased in both A8(+) and A8(−) PCOS compared to age-, weight-, and ethnicity-comparable, reproductively-normal control women. This result is consistent with the presence of similar degrees of insulin resistance in A8(+) and A8(−) PCOS women. However, 2 h post-challenge glucose levels were significantly increased in A8(+) PCOS compared to A8(−) PCOS and to control women (P<0.05, ANCOVA), as shown in Table 2 and FIG. 3. Post-challenge insulin levels did not differ in the A8(+) and A8(−) PCOS groups and were higher than in the control women; this result is consistent with the presence of insulin resistance in both A8(+) and A8(−) groups (Table 2, FIG. 3D). The adrenal androgen DHEAS tended to be higher in the A8(+) women, but this difference did not achieve statistical significance (data not shown). There were no other significant differences in hormonal parameters in the A8(+) and A8(−) PCOS groups. The A8(+) and A8(−) PCOS women who had FSIGTs were well matched for age and BMI (Table 2). They had virtually identical insulin sensitivity (SI) values indicating that they had similar degree of insulin resistance. However, glucose levels were slightly higher and insulin levels substantially lower in the A8(+) PCOS (FIG. 3A and 3B). The striking differences in insulin responses during the FSIGT are depicted by the shaded area in FIG. 3B. The observation that insulin responses to tolbutamide were much lower in A8(+) PCOS women suggests that there may be a defect in the sulfonylurea receptor. Evidence indicates that this defect may be an androgen-medicated action on K⁺_{ATP} channels in the β-cell. When expressed as area-under-the-curve (AUC) insulin:glucose, this difference approached statistical significance (P=0.09) in this very small sample of A8(+) and A8(−) PCOS women (see Table 2).

An investigation focused on whether there were phenotypic differences in A8(+) (n=19) and A8(−) (n=39) obese brothers of PCOS probands. The groups were well matched for age and BMI. Proinsulin levels, proinsulin:insulin molar ratios and TTG levels were significantly increased in A8(+) brothers compared to A8(−) brothers (see FIG. 4). HDL levels tended to be lower in A8(+) brothers (35±2 A8(+) vs 40±2 A8(−) mg/dL, P=0.053). Very few brothers had OGTTs so responses could not be analyzed. DHEAS levels tended to be higher in the A8(+) brothers (data not shown).

To determine whether A8 had an impact on metabolic parameters in normal individuals, A8(+) (n=20) and A8(−) (n=26) obese unaffected sisters as defined above were examined. There were no significant differences in fasting metabolic parameters, but too few sisters had OGTTs so changes similar to those in the PCOS sisters may have escaped detection.

Significant changes in BMI with age and in post-challenge glucose levels in A8(+) PCOS women were identified. The lack of similar increases in post-challenge insulin levels suggests a failure of compensatory insulin secretion in the A8(+) PCOS. An independent test of insulin action, the FSIGT, performed in a small subset of these A8(+) PCOS also suggests decreased insulin secretion. The magnitude of insulin resistance appears to be similar in the A8(+) and A8(−) PCOS. However, OGTT parameters of insulin action are relatively insensitive, and the sample size for the FSIGT was quite small. Accordingly, both insulin secretion as well as action needs to be assessed directly to determine the metabolic impact of A8. Further, the association of increasing BMI with age was found in cross-sectional data and needs to be confirmed in prospective studies.

The A8(+) brothers also appear to have a metabolic phenotype consistent with the insulin resistance syndrome. The increase in proinsulin levels and the proinsulin:insulin molar ratio in A8(+) brothers suggests that they may also have β-cell dysfunction. These findings are similar to the trends that were noted in the comparison of PCOS brothers to control men (see above) and now achieve statistical significance when the population is stratified by A8 status. This very important result provides a way to identify the affected brothers of PCOS women, i.e. those who are A8(+). Proinsulin levels were higher in A8(+) brothers (see FIG. 4) than in A8(+) PCOS probands (see Table 2) suggesting that there may be sex differences in the metabolic phenotype.

It was found that associations between A8 and metabolic phenotypes in PCOS women and male first-degree relatives provide strong support for the hypothesis that a gene in the region of D19S884 plays an important role in insulin action and/or secretion. There appear to be additional susceptibility genes for insulin resistance in PCOS since A8(−) PCOS women also have evidence for defects in insulin action.

Example 4

### Acquired Insulin Resistance in PCOS Skeletal Muscle

This study was performed to determine whether the defects that detected in acutely isolated skeletal muscle were intrinsic. Myoblasts were harvested from Bergstrom needle biopsies of the vastus lateralis and grown in primary culture using the method of Henry et al. There were no significant differences in population doubling time or cell number in PCOS compared to control myoblasts. There were no significant differences in the fold-stimulation of glucose transport or glucose incorporation into glycogen (see Table 3). There were significant increases in basal glucose transport (see Table 3) in PCOS compared to control. This finding may be explained by significant increases in GLUTI abundance in PCOS. GLUT4 abundance was similar in PCOS and control myotubes. Metabolic signaling pathways were similar in PCOS and control myotubes whereas mitogenic pathways were upregulated in PCOS. Basal mitogen-activated protein kinase kinase (MEK) phosphorylation tended to be increased, and insulin-stimulated MEK phosphorylation was significantly increased in PCOS without a change in MEK abundance. Consistent with this activation of MEK, p44/42 MAPK phosphorylation (detected by an antibody that recognizes p44 and p42 MAPK only when dually phosphorylated at thr202 and tyr204; the p44 and p42 bands were quantitated together) was significantly increased at baseline and in response to insulin in PCOS without any change in the abundance of these signaling proteins. There was a significant increase in p44/42 MAPK phbsphorylation at baseline in PCOS skeletal muscle biopsies (63±9 PCOS n=8 vs 30±6 n=8 control, % internal standard, P<0.05) without changes in MAPK abundance.

There were constitutive increases in glucose uptake, GLUT1 abundance and p44/42 MAPK activation in PCOS myotubes. The increase in MAPK phosphorylation in skeletal muscle biopsies indicates that the findings in cultured myotubes are not an artifact of the culture conditions. Activation of growth related MAPK pathways has not been found in other insulin resistant states and is another unique feature of the PCOS phenotype. The p44/42 MAPK pathway is activated by growth factors such as insulin and regulates cell proliferation, cell survival and gene expression. Enhanced signaling through these pathways, basally and in response to insulin, may contribute to some of the PCOS phenotype.

Example 5

### Mechanisms for Acquired Defects in Insulin Action in PCOS: Role of FFA and TNF-α in PCOS

It was concluded that decreases in IMGD and IRS-1-associated PI3-kinase activity in PCOS resolve in cultured skeletal muscle suggesting that these defects are acquired secondary to in vivo environment. Candidate factors that could modulate insulin sensitivity include androgens, FFA, TNF-α, resistin and adiponectin. Fasting FFA levels were significantly increased in obese PCOS (n=8) compared to control (n=7) women of comparable age and weight (434±46 control vs 607±58 PCOS μmol/L, P<0.05), despite higher fasting insulin levels (13±2 control vs 22±5 PCOS lU/mL) in PCOS, a finding consistent with resistance of FFA suppression by insulin in vivo. The FFA levels were similar to those in women with upper-body obesity. TNF-α levels were not significantly increased in obese PCOS (n=20) compared to weight-comparable control (n=12) women (7±2 control vs 6±1 PCOS pg/mL) in contrast to prior reports.

It is also possible that there is increased sensitivity to FFA or TNF-a actions in PCOS. To investigate this hypothesis, the impact of incubating cultured myotubes from PCOS (n=7) and control (n=7) women with the FFA palmitate (0-1 mM) for the last 48 h during the 4 d differentiation process or with TNF-α (0-25 ng/mL) for 2 h. was examined. Palmitate caused a significantly greater decrease in both basal as well as insulin-stimulated glycogen synthesis in PCOS than in control (both P<0.05), whereas TNF-α had a similar effect to decrease glycogen synthesis in PCOS and control myotubes.

The following publications are herein incorporated by reference:
1. Legro R S, D Finegood, A Dunaif. A fasting glucose to insulin ratio is a useful measure of insulin sensitivity in women with polycystic ovary syndrome. J Clin Endocrinol Metab 83:2694-2698, 1998.
2. Martens J W M, D H Geller, W Arlt, R J Auchus, V S Ossovskaya, H Rodriguez, T D Tisty, A Dunaif, W L Miller. Enzymatic activities of P450c17 stably expressed in fibroblasts from patients with the polycystic ovary syndrome. J Clin Endocrinol Metab, 85:4338-4346, 2000.
3. Rui L, V Aguirre, J K Kim, G I Shulman, A Lee, A Corbould, A Dunaif, M F White. Phosphorylation of ser307 in IRS-1 mediates feedback and heterologous inhibition of insulin signaling. J Clin Invest 107:181-189, 2001.
4. Legro R S, A R Kunselman, A Dunaif. Prevalence and predictors of dyslipidemia in women with polycystic ovary syndrome. Am J Med, 111:607-13, 2001.
5. Palmert M R, C M Gordon, A Kartashov, R S Legro, S J Emans, A Dunaif. Screening for abnormal glucose tolerance in adolescents with polycystic ovary syndrome. J Clin Endocrinol Metab, 87:1017-23, 2002.
6. Legro R S, M Urbanek, A R Kunselman, B E Leiby, A Dunaif. Self selected women with polycystic ovary syndrome are reproductively and metabolically abnormal. Fertil Steril, in press, 2002.
7. Legro R S, R Bentley-Lewis, D Driscoll, S C Wang, A Dunaif Insulin resistance in the sisters of women with polycystic ovary syndrome: association with hyperandrogenemia rather than menstrual irregularity. J Clin Endocrinol Metab, in press, 2002.
8. Franks, S, Gharani, N, Waterworth, D, Batty, S, White, D, Williamson, R, McCarthy,M: The genetic basis of polycystic ovary syndrome. Hum. Reprod. 12:2641-2648, 1997
9. Legro, R S, Spielman, R, Urbanek, M, Driscoll, D, Strauss, J F, Dunaif A: Phenotype and genotype in polycystic ovary syndrome. Recent. Prog. Horm. Res. 53:217-256, 1998
10. Carey, A H, Chan, K L, Short, F, White, D, Williamson, R, Franks, S: Evidence for a single gene effect causing polycystic ovaries and male pattern baldness. Clin. Endocrinol. (Oxf.) 38:653-658, 1993
11. Kashar-Miller, M, Azziz, R: Heritability and the risk of developing androgen excess. J. Steroid Biochem. Mol. Biol. 69:261-268, 1999
12. Lander, E S, Schork, N J: Genetic dissection of complex traits. Science 265:2037-2048, 1994
13. Legro, R S, Driscoll, D, Strauss, J F, Fox, J, Dunaif, A: Evidence for a genetic basis for hyperandrogenemia in polycystic ovary syndrome. Proc. Natl. Acad. Sci. U.S.A. 95:14956-14960, 1998
14. Urbanek, M, Legro, R S, Driscoll, D A, Azziz, R, Ehrmann, D A, Norman, R J, Strauss, J F, Spielman, R S, Dunaif, A: Thirty-seven candidate genes for polycystic ovary syndrome: strongest evidence for linkage is with follistatin. Proc. Natl. Acad. Sci. U.S.A. 96:8573-8578, 1999
15. Urbanek M, Vickery K R, Legro R S, Driscoll D A, Strauss J F, Dunaif A, Spielman R S: Two candidate genes for polycystic ovary syndrome (PCOS): follistatin and the insulin receptor (Abstract). 50th Annual Meeting, American Society of Human Genetics, Philadelphia, Pa., Abstract #1804, October 2000, 2000
16. Spielman, R S, Ewens, W J: The TDT and other family-based tests for linkage disequilibrium and association. Am. J. Hum. Genet. 59:983-989, 1996
17. Spielman, R S, McGinnis, R E, Ewens, W J: The transmission/disequilibrium test detects cosegregation and linkage. Am. J Hum. Genet. 54:559-560, 1994
18. Altshuler, D, Hirschhorn, J N, Klannemark, M, Lindgren, C M, Vohl, M C, Nemesh, J, Lane, C R, Schaffner, S F, Bolk, S, Brewer, C, Tuomi, T, Gaudet, D, Hudson, T J, Daly, M, Groop, L, Lander, E S: The common PPARgamma Pro12Ala polymorphism is associated with decreased risk of type 2 diabetes. Nat. Genet. 26:76-80, 2000
19. Tucci, S, Futterweit, W, Concepcion, E S, Greenberg, D A, Villanueva, R, Davies, T F, Tomer, Y: Evidence for association of polycystic ovary syndrome in caucasian women with a marker at the insulin receptor gene locus. Journal of Clinical Endocrinology & Metabolism 86:446-449, 2001
20. Permutt, M A, Bernal-Mizrachi, E, Inoue, H: Calpain 10: the first positional cloning of a gene for type 2 diabetes? J. Clin. Invest. 106:819-821, 2000
21. Yamagata, K, Furuta, H, Oda, N, Kaisaki, P J, Menzel, S, Cox, N J, Fajans, S S, Signorini, S, Stoffel, M, Bell, G I: Mutations in the hepatocyte nuclear factor-4alpha gene in maturity-onset diabetes of the young (MODY1). [see comments]. Nature 384:458-460, 1996
22. Herman, W H, Fajans, S S, Ortiz, F J, Smith, M J, Sturis, J, Bell, G I Polonsky, K S, Halter, J B: Abnormal insulin secretion, not insulin resistance, is the genetic or primary defect of MODY in the RW pedigree. [erratum appears in Diabetes 1994 Sep;43(9):1171]. Diabetes 43:40-46, 1994
23. Byrne, M M, Sturis, J, Fajans, S S, Ortiz, F J, Stoltz, A, Stoffel, M, Smith, M J, Bell, G I, Halter, J B, Polonsky, K S: Altered insulin secretory responses to glucose in subjects with a mutation in the MODY1 gene on chromosome 20. Diabetes 44:699-704, 1995
24. Byrne, M M, Sturis, J, Menzel, S, Yamagata, K, Fajans, S S, Dronsfield, M J, Bain, S C, Hattersley, A T, Velho, G, Froguel, P, Bell, G I, Polonsky, K S: Altered insulin secretory responses to glucose in diabetic and nondiabetic subjects with mutations in the diabetes susceptibility gene MODY3 on chromosome 12. Diabetes 45:1503-1510, 1996
25. Vionnet, N, Hani, E-H, Dupont, S, Gallina, S, Francke, S, Dotte, S, DeMatos, F, Durand, E, Lepretre, F, Lecoeur, C, Gallina, P, Zekiri, L, Dina, C, Froguel, P: Genomewide search for type 2 diabetes-susceptibility genes in French whites: evidence for a novel susceptibility locus for early-onset diabetes on chromosome 3q27-qter and independent replication of a type 2-diabetes locus on chromosome 1q21 -q24. Am. J. Hum. Genet. 2000 67:1470-1480, 2000
26. Watanabe, R M, Ghosh, S, Langefeld, C D, Valle, T T, Hauser, E R, Magnuson, V L, Mohlke, K L, Silander, K, Ally, D S, Chines, P, Blaschak-Harvan, J, Douglas, J A, Duren, W L, Epstein, M P, Fingerlin, T E, Kaleta, H S, Lange, E M, Li, C, McEachin, R C, Stringham, H M, Trager, E, White, P P, Balow, Jr J, Birznieks, G, Chang, J, Eldridge, W: The Finland-United States Investigation of non-insulin-dependent diabetes mellitus genetics (FUSION) study. II. An autosomal genome scan for diabetes-related quantitative-trait loci. Am. J. Hum. Genet.2000 67:1186-1200, 2000
27. Waterworth, D M, Bennett, S T, Gharani, N, McCarthy, W, Hague, S, Batty, S, Conway, G S, White, D, Todd, J A, Franks, S, Williamson, R: Linkage and association of insulin gene VNTR regulatory polymorphism with polycystic ovary syndrome. Lancet 349:986-990, 1997
28. Bennett, S T, Todd, J A, Waterworth, D M, et al.: Association of insulin gene VNTR polymorphism with polycystic ovary syndrome. Lancet 349:1771-1772, 1997
29. Eaves, I A, Bennett, S T, Forster, P, Ferber, K M, Ehrmann, D A, Wilson, A J, Bhattacharyya, S, Ziegler, A G, Brinkmann, B, Todd, J A: Transmission radio distortion at the INS-IGF2 VNTR. Nat. Genet. 22:324-325, 1999
30. Urbanek, M, Wu, X, Vickery, K R, Kao, L C, Christenson, L K, Schneyer, A, Legro, R S, Driscoll, D A, Strauss, J F, Dunaif, A, Spielman, R S: Allelic variants of the follistatin gene in polycystic ovary syndrome. Journal of Clinical Endocrinology & Metabolism 85:4455-4461, 2000
31. El Mkadem, S A, Lautier, C, Macari, F, Molinari, N, Lefebvre, P, Renard, E, Gris, J C, Cros, G, Daures, J P, Bringer, J, White, M F, Grigorescu, F: Role of allelic variants Gly972Arg of IRS-1 and Gly1057Asp of IRS-2 in moderate-to-severe insulin resistance of women with polycystic ovary syndrome. Diabetes 50:2164-2168, 2001
32. Hara, M, Alcoser, S Y, Qaadir, A, Beiswenger, K K, Cox, N J, Ehrmann, D A: Insulin resistance is attenuated in women with polycystic ovary syndrome with the Pro(12)Ala polymorphism in the PPARgamma gene. Journal of Clinical Endocrinology & Metabolism 87:772-775, 2002
33. Almind, K, Doria, A, Kahn, C R: Putting the genes for type II diabetes on the map. Nature Medicine 7:277-279, 2001
34. Elbein, S C: Genetics of type 2 diabetes: an overview for the millennium. [Review] [28 refs]. Diabetes Tecinology & Therapeutics 2:391-400, 2000
35. Barsh, G S, Farooqi, I S, O'Rahilly, S: Genetics of body-weight regulation. [Review] [125 refs]. Nature 404:644-651, 2000
36. Dunaif, A: Insulin resistance and the polycystic ovary syndrome: mechanism and implications for pathogenesis. Endocr. Rev. 18:774-800, 1997
37. Dunaif, A, Thomas, A: Current Concepts in the Polycystic Ovary Syndrome. Ann. Rev. Med.401-419, 2001
38. Dunaif, A, Diamanti-Kandarakis, E, Lee, A, Wu, X: Defects in insulin receptor signaling in vivo in the polycystic ovary syndrome (PCOS). Am. J. Physiol. 281 :E392-E399, 2001
39. Dunaif, A, Xia, J, Book, C B, Schenker, E, Tang, Z: Excessive insulin receptor serine phosphorylation in cultured fibroblasts and in skeletal muscle. A potential mechanism for insulin resistance in the polycystic ovary syndrome. J. Clin. Invest. 96:801-810, 1995
40. Li M, Youngren J, Maddux B A, Dunaif A, Thomas A, Zhang B, Goldfine I: Factors extrinsic to the insulin receptor cause insulin resistance in muscle and fibroblasts from PCOS. (Abstract). 82nd Annual Meeting of the Endocrine Society, Toronto, Canada Abstract 2331: June 2000, 2000
41. Book, C B, Dunaif, A: Selective insulin resistance in the polycystic ovary syndrome. J. Clin. Endocrinol. Metab. 84:3110-3116, 1999
42. DeFronzo, R A: Lilly lecture 1987. The triumvirate: beta-cell, muscle, liver. A collusion responsible for NIDDM. Diabetes 37:667-687, 1988
43. Henry, R R, Ciaraldi, T P, Abrams-Carter, L, Mudaliar, S, Park, K S, Nikoulina, S E: Glycogen synthase activity is reduced in cultured skeletal muscle cells of non-insulin-dependent diabetes mellitus subjects. Biochemical and molecular mechanisms. J. Clin. Invest. 98:1231-1236, 1996
44. Henry, R R, Abrams, L, Nikoulina, S, Ciaraldi, T P: Insulin action and glucose metabolism in nondiabetic control and NIDDM subjects. Comparison using human skeletal muscle cell cultures. Diabetes 44:936-946, 1995
45. Cusi, K, Maezono, K, Osman, A, Pendergrass, M, Patti, M E, Pratipanawatr, T, DeFronzo, R A, Kahn, C R, Mandarino, L J: Insulin resistance differentially affects the PI 3-kinase- and MAP kinase-mediated signaling in human muscle. J. Clin. Invest. 105:311-320, 2000
46. Kido, Y, Burks, D J, Withers, D, Bruning, J C, Kahn, C R, White, M F, Accili, D: Tissue-specific insulin resistance in mice with mutations in the insulin receptor, IRS-1, and IRS-2. J. Clin. Invest. 105:199-205, 2000
47. McAllister J M, Cocozza S: Altered mitogen activated protein kinase (MAPK) signaling in theca and granulosa cells isolated from patients with polycystic ovary syndrome (PCOS) (Abstract). 83rd Annual Meeting Endo. Society, 2001
48. Ciaraldi, T P, Morales, A J, Hickman, M G, Odom-Ford, R, Yen, S S, Olefsky, J M: Lack of insulin resistance in fibroblasts from subjects with polycystic ovary syndrome. Metabolism 47:940-946, 1998
49. Kahn, B B, Flier, J S: Obesity and insulin resistance. J. Clin. Invest. 106:473-481, 2000
50. Yamauchi, T, Kamon, J, Waki, H, Terauchi, Y, Kubota, N, Hara, K, Mori, Y, Ide, T, Murakami, K, Tsuboyama-Kasaoka, N, Ezaki, O, Akanuma, Y, Gavrilova, O, Vinson, C, Reitman, M L, Kagechika, H, Shudo, K, Yoda, M, Nakano, Y, Tobe, K, Nagai, R, Kimura, S, Tomita, M, Froguel, P, Kadowaki, T: The fat-derived hormone adiponectin reverses insulin resistance associated with both lipoatrophy and obesity. [see comments]. Nature Medicine 7:941-946, 2001
51. Abel, E D, Peroni, O, Kim, J K, Kim, Y B, Boss, O, Hadro, E, Minnemann, T, Shulman, G I, Kahn, B B: Adipose-selective targeting of the GLUT4 gene impairs insulin action in muscle and liver. [see comments]. Nature 409:729-733, 2001
52. Greco, A V, Mingrone, G, Giancaterini, A, Manco, M, Morroni, M, Cinti, S, Granzotto, M, Vettor, R, Camastra, S, Ferrannini, E: Insulin resistance in morbid obesity: reversal with intramyocellular fat depletion. Diabetes 51:144-151, 2002
53. Wu X, Eisner J R, Dunaif A: Evidence for increased sensitivity to the insulin-resistance inducing actions of free fatty acids in cultured polycystic ovary syndrome skeletal muscle (Abstract). 61st Scientific Sessions of the American Diabetes Association, June 2001, Philadelphia, Pa., Abstract 1107-P, 2001
54. Kissebah, A H, Peiris, A N: Biology of regional body fat distribution: relationship to non-insulin- dependent diabetes mellitus. Diabetes Metab Rev. 5:83-109, 1989
55. Peiris, A N, Mueller, R A, Struve, M F, Smith, G A, Kissebah, A H: Relationship of androgenic activity to splanchnic insulin metabolism and peripheral glucose utilization in premenopausal women. J Clin Endocrinol Metab 64:162-169, 1987
56. Kirschner, M A, Samojlik, E, Drejka, M, Szmal, E, Schneider, G, Ertel, N: Androgen-estrogen metabolism in women with upper body versus lower body obesity. Journal of Clinical Endocrinology & Metabolism 70:473479, 1990
57. Dunaif, A, Green, G, Futterweit, W, Dobrjansky, A: Suppression of hyperandrogenism does not improve peripheral or hepatic insulin resistance in the polycystic ovary syndrome. J. Clin. Endocrinol. Metab. 70:699-704, 1990

58. Moghetti, P, Tosi, F, Castello, R, Magnani, C M, Negri, C, Brun, E, Furlani, L, Caputo, M, Muggeo, M: The insulin resistance in women with hyperandrogenism is partially reversed by antiandrogen treatment: evidence that androgens impair insulin action in women. J Clin Endocrinol Metab 81:952-960, 1996
59. Dumesic, D A, Abbott, D H, Eisner, J R, Herrmann, R R, Reed, J E, Welch, T J, Jensen, M D: Pituitary desensitization to gonadotropin-releasing hormone increases abdominal adiposity in hyperandrogenic anovulatory women. Fertil. Steril. 70:94-101, 1998
60. Ek, I, Arner, P, Bergqvist, A, Carlstrom, K, Wahrenberg, H: Impaired adipocyte lipolysis in nonobese women with the polycystic ovary syndrome: a possible link to insulin resistance? J. Clin. Endocinol. Metab. 82:1147-1153, 1997
61. Holte, J: Polycystic ovary syndrome and insulin resistance: thrifty genes struggling with over-feeding and sedentary life style? J Endocrinol Invest. 21:589-601, 1998
62. Holte, J, Bergh, T, Berne, C, Wide, L, Lithell, H: Restored insulin sensitivity but persistently increased early insulin secretion after weight loss in obese women with polycystic ovary syndrome. J. Clin. Endocrinol. Metab. 80:2586-2593, 1995
63. Dumesic, D A, Nielsen, M F, Abbott, D H, Eisner, J R, Nair, K S, Rizza, R A: Insulin action during variable hyperglycemic-hyperinsulinemic infusions in hyperandrogenic anovulatory patients and healthy women. Fertil. Steril. 72:458-466, 1999
64. Dunaif, A, Segal, K R, Shelley, D R, Green, G, Dobrjansky, A, Licholai, T: Evidence for distinctive and intrinsic defects in insulin action in polycystic ovary syndrome. Diabetes 41:1257-1266, 1992
65. Dunaif, A, Segal, K R, Futterweit, W, Dobrjansky, A: Profound peripheral insulin resistance, independent of obesity, in polycystic ovary syndrome. Diabetes 38:1165-1174,1989
66. Carey, D G, Jenkins, A B, Campbell, L V, Freund, J, Chisholm, D J: Abdominal fat and insulin resistance in normal and overweight women: Direct measurements reveal a strong relationship in subjects at both low and high risk of NIDDM. Diabetes 45:633-638, 1996
67. Vicennati, V., Gambineri, A., Calzoni, F., Casimirri, F., Macor, C., Vettor, R., and Pasquali, R. Serum Leptin in Obese Women with Polycystic Ovary Syndrome is Correlated With Body Weight and Fat Distribution But Not With Androgen and Insulin Levels. Metabolism 47(8), 988-992.1998. Ref Type: Generic
68. Pasquali, R, Gambineri, A, Biscotti, D, Vicennati, V, Gagliardi, L, Colitta, D, Fiorini, S, Cognigni, G E, Filicori, M, Morselli-Labate, A M: Effect of long-term treatment with metformin added to hypocaloric diet on body composition, fat distribution, and androgen and insulin levels in abdominally obese women with and without the polycystic ovary syndrome. J. Clin. Endocrinol. Metab. 85:2767-2774,2000
69. Eisner, J R, Dumesic, D A, Kemnitz, J W, Abbott, D H: Timing of prenatal androgen excess determines differential impairment in insulin secretion and action in adult female rhesus monkeys. J. Clin. Endocrinol. Metab. 85:1206-1210, 2000
70. Bergman, R N, Mittelman, S D: Central role of the adipocyte in insulin resistance. J. Basic. Clin. Physiol. Pharmzacol. 9:205-221, 1998
71. Boden, G: Role of fatty acids in the pathogenesis of insulin resistance and NIDDM. [erratum appears in Diabetes 1997 Mar;46(3):536]. Diabetes 46:3-10, 1997
72. Frias, J P, Macaraeg, G B, Ofrecio, J, Yu, J G, Olefsky, J M, Kruszynska, Y T: Decreased susceptibility to fatty acid-induced peripheral tissue insulin resistance in women. Diabetes 50:1344-1350, 2001
73. Holte, J, Bergh, T, Berne, C, Lithell, H: Serum lipoprotein lipid profile in women with the polycystic ovary syndrome: relation to anthropometric, endocrine and metabolic variables. Clin. Endocrinol. (Oxf.) 41:463-471, 1994
74. Ek, I, Arner, P, Ryden, M, Holm, C, Thorne, A, Hoffstedt, J, Wahrenberg, H: A unique defect in the regulation of visceral fat cell lipolysis in the polycystic ovary syndrome as an early link to insulin resistance. Diabetes 51 :484-492, 2002
75. Godfrey, K M: Maternal regulation of fetal development and health in adult life. [Review] [88 refs]. European Journal of Obstetrics, Gynecology, & Reproductive Biology 78:141-150, 1998
76. Breier, B H, Vickers, M H, Ikenasio, B A, Chan, K Y, Wong, W P: Fetal programming of appetite and obesity. Molecular & Cellular Endocrinology 185:73-79, 2001
77. Young, L E, Fernandes, K, McEvoy, T G, Butterwith, S C, Gutierrez,C G, Carolan, C, Broadbent, P J, Robinson, J J, Wilmut, I, Sinclair, K D: Epigenetic change in IGF2R is associated with fetal overgrowth after sheep embryo culture. Nature Genetics 27:153-154,2001
78. Godfrey, K M, Barker, D J: Fetal programming and adult health. [Review] [70 refs]. Public Health Nutrition 4:611-624, 2001
79. Susser, M, Levin, B: Ordeals for the fetal programming hypothesis. The hypothesis largely survives one ordeal but not another. [letter; comment.] [see comments.]. BMJ 318:885-886, 1999
80. Silverman, B L, Rizzo, T A, Cho, N H, Metzger, B E: Long-term effects of the intrauterine environment. The Northwestern University Diabetes in Pregnancy Center. Diabetes Care 21 Suppl2:B142-B149,1998
81. Lonnqvist, F, Thorne, A, Large, V, Amer, P: Sex differences in visceral fat lipolysis and metabolic complications of obesity. Arterioscler. Thromb. Vasc.Biol. 17:1472-1480, 1997
82. Reiling, B A, Drackley, J K, Grum, L R, Berger, L L: Effects of prenatal androgenization and lactation on adipose tissue metabolism in finishing single-calf heifers. Journal of Animal Science 75:1504-1513, 1997
83. Eisner, J R, Barnett, M A, Dumesic, D A, Abbott, D H: Ovarian hyperandrogenism in adult female rhesus monkeys exposed to prenatal androgen excess. Fertility & Sterility 77:167-172, 2002
84. Dumesic, D A, Abbott, D H, Eisner, J R, Goy, R W: Prenatal exposure of female rhesus monkeys to testosterone propionate increases serum luteinizing hormone levels in adulthood. Fertil. Steril. 67:155-163, 1997
85. Nilsson, C, Niklasson, M, Eriksson, E, Bjorntorp, P, Holmang, A: Imprinting of female offspring with testosterone results in insulin resistance and changes in body fat distribution at adult age in rats. J. Clin. Invest. 101:74-78, 1998
86. Ibanez, L, Potau,N, Zampolli, M, Prat, N, Virdis, R, Vicens-Calvet, E, Carrascosa, A: Hyperinsulinemia in postpubertal girls with a history of premature pubarche and functional ovarian hyperandrogenism. Journal of Clinical Endocrinology & Metabolism 81:1237-1243, 1996
87. Ibanez, L, Potau, N, Francois, I, de Zegher, F: Precocious pubarche, hyperinsulinism, and ovarian hyperandrogenism in girls: relation to reduced fetal growth. Journal of Clinical Endocrinology & Metabolism 83:3558-3562, 1998
88. Dunaif, A, Sorbara, L, Delson, R, Green, G: Ethnicity and polycystic ovary syndrome are associated with independent and additive decreases in insulin action in Caribbean-Hispanic women. Diabetes 42:1462-1468, 1993
89. Legro R S, Bentley-Lewis, R, Driscoll, A, Wang S, Dunaif A: Hyperinsulininemia in the sisters of women with polycystic ovary syndrome: association with hyperandrogenemia rather than menstrual irregularity. J Clin Endominol Metab, in press, 2002
90. Legro R S, Bentley-Lewis, R, Driscoll A, Wang, S C, Dunaif A: Elevated dehydroepiandrosterone sulfate levels as the reproductive phenotype in the brothers of women with polycystic ovary syndrome. J Clin Endocrinol Metab, in press, 2002
91. Strachan T, Read A P: Genetic mapping of complex characters. In Human Molecular Genetics 2. Oxford, John Wiley & Sons, Inc.—BIOS Scientific Publishers Ltd, 1999, p. 283-294
92. Strachan T, Read A P: Human Molecular Genetics 2nd Edition. Oxford, UK, BIOS Scientific Publishers Ltd., 1999,
93. Nomiyama, H, Amano, K, Kusuda, J, Imai, T, Miura, R, Yoshie, O, Matsuda, Y: The human CC chemokine TECK (SCYA25) maps to chromosome 19p13.2. Genomics 51:311-312, 1998
94. Foltz, I N, Gerl, R E, Wieler, J S, Luckach, M, Salmon, R A, Schrader, J W: Human nitrogen-activated protein kinase kinase 7 (MKK7) is a highly conserved c-Jun N-terminal kinase/stress-activated protein kinase (JNK/SAPK) activated by environmental stresses and physiological stimuli. Journal of Biological Chemistry 273:9344-9351, 1998
95. Steppan, C M, Bailey, S T, Bhat, S, Brown, E J, Banerjee, R R, Wright, C M, Patel, H R, Ahima, R S, Lazar, M A: The hormone resistin links obesity to diabetes. [see comments]. Nature 409:307-312, 2001
96. Yamagata, K, Oda, N, Kaisaki, P J, Menzel, S, Furuta, H, Vaxillaire, M, Southam, L, Cox, R D, Lathrop, G M, Boriraj, V V, Chen, X, Cox, N J, Oda, Y, Yano, H, Le Beau, M M, Yamada, S, Nishigori, H, Takeda, J, Fajans, S S, Hattersley, A T, Iwasaki, N, Hansen, T, Pedersen, O, Polonsky, K S, Bell, G I: Mutations in the hepatocyte nuclear factor-1 alpha gene in maturity-onset diabetes of the young (MODY3). [see comments]. Nature 384:455-458, 1996
97. Horikawa, Y, Oda, N, Cox, N J, Li, X, Orho-Melander, M, Hara, M, Hinokio, Y, Lindner, T H, Mashima, H, Schwarz, P E, del Bosque-Plata, L, Horikawa, Y, Oda, Y, Yoshiuchi, I, Colilla, S, Polonsky, K S, Wei, S, Concannon, P, Iwasaka, N, Schulze, J, Baier, L J, Bogardus, C, Groop, L, Boerwinkle, E, Hanis, C L, Bell, G I: Genetic variation in the gene encoding calpain-10 is associated with type 2 diabetes mellitus. Nat. Genet. 26:163-175, 2000
98. Aguilar-Bryan, L, Bryan, J, Nakazaki M: Of mice and men: K(ATP) channels and insulin secretion. Recent Progress in Hormone Research 56:47-68, 2001
99. DeFronzo R A, F E: Insulin resistance. A multifaceted syndrome responsible for NIDDM, obesity, hypertension, dyslipidemia, and atherosclerotic cardiovascular disease. Diabetes Care 14:173-194, 1991
100. Haffner, S M, Mykkanen, L, Valdez, R A, Stern, M P, Holloway, D L, Monterrosa, A, Bowsher, R R: Disproportionately increased proinsulin levels are associated with the insulin resistance syndrome. J. Clin. Endocrinol. Metab. 79:1806-1810, 1994
101. Kahn, S E, Leonetti, D L, Prigeon, R L, Boyko, E J, Bergstrom, R W, Fujimoto, W Y: Proinsulin as a marker for the development of NIDDM in Japanese-American men. Diabetes 44:173-179, 1995
102. Haffier, S M, Bowsher, R R, Mykkanen, L, Hazuda, H P, Mitchell, B D, Valdez, R A, Gingerich, R, Monterossa, A, Stem, M P: Proinsulin and specific insulin concentration in high- and low-risk populations for NIDDM. Diabetes 43:1490-1493, 1994
103. Ciaraldi, T P, Abrams, L, Nikoulina, S, Mudaliar, S, Henry, R R: Glucose transport in cultured human skeletal muscle cells. Regulation by insulin and glucose in nondiabetic and non-insulin-dependent diabetes mellitus subjects. J. Clin. Invest. 96:2820-2827, 1995
104. Henry, R R, Ciaraldi, T P, Mudaliar, S, Abrams, L, Nikoulina, S E: Acquired defects of glycogen synthase activity in cultured human skeletal muscle cells: influence of high glucose and insulin levels. Diabetes 45:400-407, 1996
105. Ciaraldi, T P, Carter, L, Nikoulina, S, Mudaliar, S, McClain, D A, Henry, R R: Glucosamine regulation of glucose metabolism in cultured human skeletal muscle cells: divergent effects on glucose transport/phosphorylation and glycogen synthase in non-diabetic and type 2 diabetic subjects. Endocrinology 140:3971-3980, 1999
106. Cohen P: The search for physiological substrates of MAP and SAP kinases in mammalian cells. Trends. Cell Biol. 7:353-361, 1997
107. Martin-Blanco E: p38 MAPK signalling cascades: ancient roles and new functions. Bioessays 22:637-645, 2001
108. Jensen, M D, Haymond, M W, Rizza, R A, Cryer, P E, Miles, J M: Influence of body fat distribution on free fatty acid metabolism in obesity. J. Clin. Invest. 83:1168-1173, 1989
109. Naz, R K, Thurston, D, Santoro, N: Circulating tumor necrosis factor (TNF)-alpha in normally cycling women and patients with premature ovarian failure and polycystic ovaries. Am. J. Reprod. Immunol. 34:170-175, 1995
110. Gonzalez, F, Thusu, K, Abdel-Rahman, E, Prabhala, A, Tomani, M, Dandona, P: Elevated serum levels of tumor necrosis factor alpha in normal-weight women with polycystic ovary syndrome. Metabolism: Clinical & Experimental 48:437-441, 1999
111. Schmitz-Peiffer, C, Craig, D L, Biden, T J: Cerarnide generation is sufficient to account for the inhibition of the insulin-stimulated PKB pathway in C2C12 skeletal muscle cells pretreated with palmitate. J. Biol. Chem. 274:24202-24210, 1999
112. Saghizadeh M, Ong, J M, Garvey, W T, Henry, R R, Kem, P A: The expression of TNF alpha by human muscle. Relationship to insulin resistance. J. Clin. Invest. 97:1111-1116, 1996
113. Ranganathan, S, Davidson, M B: Effect of tumor necrosis factor-alpha on basal and insulin-stimulated glucose transport in cultured muscle and fat cells. Metabolism 45:1089-1094, 1996
114. Storz, P, Doppler, H, Wernig, A, Pfizenmaier, K, Muller, G: Cross-talk mechanisms in the development of insulin resistance of skeletal muscle cells palmitate rather than tumour necrosis factor inhibits insulin-dependent protein kinase B (PKB)/Akt stimulation and glucose uptake. Eur. J. Biochem. 266:17-25, 1999
115. Frisancho A R: Anthropometric standards for the assessment of growth and nutritional status. The University of Michigan Press, 1990,
116. Xu, X, De Pergola, G, Bjomtorp P: The effects of androgens on the regulation of lipolysis in adipose precursor cells. Endocrinology 126:1229-1234, 1990
117. Krakower, G R, Meier, D A, Kissebah, A H: Female sex hormones, perinatal, and peripubertal androgenization on hepatocyte insulin dynamics in rats. Am. J. Physiol. 264:E342-E347, 1993
118. Fajans, S S, Bell,G I, Polonsky,K S: Molecular mechanisms and clinical pathophysiology of maturity-onset diabetes of the young. [Review] [68 refs]. New England Journal of Medicine 345:971-980, 2001
119. DeFronzo, R A, Tobin, J D, Andres, R: Glucose clamp technique: a method for quantifying insulin secretion and resistance. Am. J. Physiol. 237:E214-E223, 1979
120. Bergman, R N: New concepts in extracellular signaling for insulin action: the single gateway hypothesis. Recent. Prog. Horm. Res. 52:359-385, 1997
121. Ukkola, O, Tremblay, A, Bouchard, C: Beta-2 adrenergic receptor variants are associated with subcutaneous fat accumulation in response to long-term overfeeding. International Journal of Obesity 25:1604-1608, 2001
122. Hughes, V A, Fiatarone, M A, Fielding, R A, Kahn, B B, Ferrara, C M, Shepherd P, Fisher, E C, Wolfe, R R, Elahi, D, Evans, W J: Exercise increases muscle GLUT-4 levels and insulin action in subjects with impaired glucose tolerance. Am. J. Physiol. 264:E855-E862,1993
123. Cline, G W, Petersen, K F, Krssak, M, Shen, J, Hundal, R S, Trajanoski, Z, Inzucchi, S, Dresner, A, Rothman, D L, Shulman, G I: Impaired glucose transport as a cause of decreased insulin-stimulated muscle glycogen synthesis in type 2 diabetes. N. Engl. J Med. 341:240-246, 1999
124. Albu, J B, Curi, M, Shur, M, Murphy, L, Matthews, D E, Pi-Sunyer, F X: Systemic resistance to the antilipolytic effect of insulin in black and white women with visceral obesity. Am. J. Physiol. 277:E551-E560, 1999
125. Morales, A J, Laughlin, G A, Butzow, T, Maheshwari, H, Baumann, G, Yen, S S: Insulin, somatotropic, and luteinizing hormone axes in lean and obese women with polycystic ovary syndrome: common and distinct features. J Clin Endocrinol Metab 81:2854-2864, 1996
126. Nielsen, S, ller, N, Christiansen, J S, rgensen, J O: Pharmacological antilipolysis restores insulin sensitivity during growth hormone exposure. Diabetes 50:2301-2308, 2001
127. Bergman, R N: Lilly lecture 1989. Toward physiological understanding of glucose tolerance. Minimal-model approach. Diabetes 38:1512-1527, 1989
128. Dunaif, A, Finegood, D T: Beta-cell dysfunction independent of obesity and glucose intolerance in the polycystic ovary syndrome. J. Clin. Endocrinol. Metab. 81:942-947, 1996
129. Weyer, C, Bogardus, C, Pratley, R E: Metabolic characteristics of individuals with impaired fasting glucose and/or impaired glucose tolerance. Diabetes 48:2197-2203, 1999
130. Weyer, C, Pratley, R E, Salbe, A D, Bogardus, C, Ravussin, E, Tataranni, P A: Energy expenditure, fat oxidation, and body weight regulation: a study of metabolic adaptation to long-term weight change. Journal of Clinical Endocrinology & Metabolism 85:1087-1094, 2000
131. Weyer, C, Bogardus, C, Mott, D M, Pratley, R E: The natural history of insulin secretory dysfunction and insulin resistance in the pathogenesis of type 2 diabetes mellitus. Journal of Clinical Investigation 104:787-794, 1999
132. Bergman, R N, Hope, I D, Yang, Y J, Watanabe, R M, Meador, M A, Youn, J H, Ader, M: Assessment of insulin sensitivity in vivo: a critical review. Diabetes Metab. Rev. 5:411-429, 1989
133. Tchernof, A, Starling, R D, Turner, A, Shuldiner, A R, Walston, J D, Silver, K, Poehlman, E T: Impaired capacity to lose visceral adipose tissue during weight reduction in obese postmenopausal women with the Trp64Arg beta3-adrenoceptor gene variant. Diabetes 49:1709-1713, 2000
134. Ward, W K, Bolgiano, D C, McKnight, B, Halter, J B, Porte, D, Jr.: Diminished B cell secretory capacity in patients with noninsulin-dependent diabetes mellitus. Journal of Clinical Investigation 74:1318-1328, 1984
135. Kahn, S E, Prigeon, R L, Schwartz, R S, Fujimoto, W Y, Knopp R H, Brunzell, J D, Porte, D, Jr.: Obesity, body fat distribution, insulin sensitivity and Islet beta-cell function as explanations for metabolic diversity. [Review] [50 refs]. Journal of Nutrition 131:354S-360S, 2001
136. Porte, D, Jr., Kahn, S E: beta-cell dysfunction and failure in type 2 diabetes: potential mechanisms. [Review] [35 refs]. Diabetes 50 Suppl 1:S160-S163, 2001
137. Larsson, H, Ahren, B: Glucose-dependent arginine stimulation test for characterization of islet function: studies on reproducibility and priming effect of arginine. Diabetologia 41:772-777, 1998
138. Larsson, H, Berglund, G, Ahren, B: Glucose modulation of insulin and glucagon secretion is altered in impaired glucose tolerance. Journal of Clinical Endocrinology & Metabolism 80:1778-1782, 1995
139. Pratley R E: The role of impaired early insulin secretion in the pathogenesis of type II diabetes mellitus. Diabetologia 44:929-945, 2001
140. Orskov, C: Glucagon-like peptide-1, a new hormone of the entero-insular axis. [Review] [122 refs]. Diabetologia 35:701-711, 1992
141. Fritsche, A, Stefan, N, Hardt, E, Haring, H, Stumvoll, M: Characterisation of beta-cell dysfunction of impaired glucose tolerance: evidence for impairment of incretin-induced insulin secretion. Diabetologia 43:852-858, 2000
142. Butler, P C, Chou, J, Carter, W B, Wang, Y N, Bu, B H, Chang, D, Chang, J K, Rizza, R A: Effects of meal ingestion on plasma amylin concentration in NIDDM and nondiabetic humans. Diabetes 39:752-756, 1990
143. Hovorka, R, Chassin, L, Luzio, S D, Playle, R, Owens, D R: Pancreatic beta-cell responsiveness during meal tolerance test: model assessment in normal subjects and subjects with newly diagnosed noninsulin-dependent diabetes mellitus. Journal of Clinical Endocrinology & Metabolism 83:744-750, 1998
144. Cavaghan, M K, Ehrmann, D A, Polonsky, K S: Interactions between insulin resistance and insulin secretion in the development of glucose intolerance. [Review] [38 refs]. Journal of Clinical Investigation 106:329-333, 2000
145. Polonsky, K S: Lilly Lecture 1994. The beta-cell in diabetes: from molecular genetics to clinical research. Diabetes 44:705-717, 1995
146. Van Cauter, E, Mestrez, F, Sturis, J, Polonsky, K S: Estimation of insulin secretion rates from C-peptide levels. Comparison of individual and standard kinetic parameters for C-peptide clearance. Diabetes 41:368-377, 1992
147. O'Meara, N M, Blackman, J D, Ehrmann, D A, Barnes, R B, Jaspan, J B, Rosenfield, R L, Polonsky, K S: Defects in beta-cell function in functional ovarian hyperandrogenism. J. Clin. Endocrinol. Metab. 76:1241-1247, 1993
148. Ehrmann, D A, Sturis, J, Byme, M M, Karrison, T, Rosenfield, R L, Polonsky, K S: Insulin secretory defects in polycystic ovary syndrome. Relationship to insulin sensitivity and family history of non-insulin-dependent diabetes mellitus. J Clin Invest. 96:520-527, 1995
149. Larsson, H, Ahren, B: Relative hyperproinsulinemia as a sign of islet dysfunction in women with impaired glucose tolerance. Journal of Clinical Endocrinology & Metabolism 84:2068-2074, 1999
150. Herman, W H, Fajans, S S, Smith, M J, Polonsky, K S, Bell, G I, Halter, J B: Diminished insulin and glucagon secretory responses to arginine in nondiabetic subjects with a mutation in the hepatocyte nuclear factor-4alpha/MODY1 gene. Diabetes 46:1749-1754, 1997
151. Larsson, H, Ahren, B: Glucose intolerance is predicted by low insulin secretion and high glucagon secretion: outcome of a prospective study in postmenopausal Caucasian women. Diabetologia 43:194-202, 2000
152. Bergman, R N, Phillips, L S, Cobelli, C: Physiologic evaluation of factors controlling glucose tolerance in man: measurement of insulin sensitivity and beta-cell glucose sensitivity from the response to intravenous glucose. J. Clin. Invest. 68:1456-1467, 1981
153. Corcoran M H, Breda E, Cavaghan M K, Imperial J,. Polonsky K S, Ehrmann D A: Impaired beta-cell compensation to dexamethasone-induced hyperglycemia in women with polycystic ovary syndrome (PCOS) (Abstract). 82nd Annual Meeting of the Endocrine Society, June 2000, Toronto, Canada, Abstract 1651 Abstract 1651: June 2000, 2000
154. Barbieri, R L, Makris, A, Ryan, K J: Effects of insulin on steroidogenesis in cultured porcine ovarian theca. Fertil. Steril. 40:237-241, 1983
155. Watanabe, R M, Bergman, R N: Accurate measurement of endogenous insulin secretion does not require separate assessment of C-peptide kinetics. Diabetes 49:373-382,2000
156. Bergman, R N, Watanabe, R, Rebrin, K, Ader, M, Steil, G: Toward an integrated phenotype in pre-NIDDM. Diabet. Med. 13:S67-S77, 1996
157. Perfetti, R, Merkel, P: Glucagon-like peptide-1: a major regulator of pancreatic beta-cell function. [Review] [56 refs]. European Journal of Endocrinology 143:717-725, 2000
158. Porte, D, Jr.: Clinical importance of insulin secretion and its interaction with insulin resistance in the treatment of type 2 diabetes mellitus and its complications. [Review] [85 refs]. Diabetes/Metabolism Research Reviews 17:181-188, 2001
159. Mitrakou, A, Vuorinen-Markkola, H, Raptis, G, Toft, I, Mokan, M, Strumph, P, Pimenta, W, Veneman, T, Jenssen, T, Bolli, G: Simultaneous assessment of insulin secretion and insulin sensitivity using a hyperglycemia clamp. Journal of Clinical Endocrinology & Metabolism 75:3 79-3 82, 1992
160. Kjems, L L, Kirby, B M, Welsh, E M, Veldhuis, J D, Straume, M, McIntyre, S S, Yang, D, Lefebvre, P, Butler, P C: Decrease in beta-cell mass leads to impaired pulsatile insulin secretion, reduced postprandial hepatic insulin clearance, and relative hyperglucagonemia in the minipig. Diabetes 50:2001-2012, 2001
161. Boden, G, Chen, X, Polansky, M: Disruption of circadian insulin secretion is associated with reduced glucose uptake in first-degree relatives of patients with type 2 diabetes. Diabetes 48:2182-2188, 1999
162. Fritsche, A, Stefan, N, Hardt, E, Schutzenauer, S, Haring, H, Stumvoll, M: A novel hyperglycaemic clarnp for characterization of islet function in humans: assessment of three different secretagogues, maximal insulin response and reproducibility. European Journal of Clinical Investigation 30:411-418, 2000
163. Winter, J S, Faiman, C, Reyes, F I: Sex steroid production by the human fetus: its role in morphogenesis and control by gonadotropins. [Review] [62 refs]. Birth Defects: Original Article Series 13:41-58, 1977
164. Nagamani, M, McDonough, P G, Ellegood, J O, Mahesh, V B: Maternal and amniotic fluid steroids throughout human pregnancy. American Journal of Obstetrics & Gynecology 134:674-680, 1979
165. Robinson, J D, Judd, H L, Young, P E, Jones, O W, Yen, S S: Amniotic fluid androgens and estrogens in midgestation. Journal of Clinical Endocrinology & Metabolism 45:755-761, 1977
166. Forest, M G, de Peretti, E, Lecoq, A, Cadillon, E, Zabot, M T, Thoulon, J M: Concentration of 14 steroid hormones in human amniotic fluid of midpregnancy. Journal of Clinical Endocrinology & Metabolism 51:816-822, 1980
167. Simmons, D, France, J T, Keelan, J A, Song, L, Knox, B S: Sex differences in umbilical cord serum levels of inhibin, testosterone, oestradiol, dehydroepiandrosterone sulphate, and sex hormone-binding globulin in human term neonates. Biology of the Neonate 65:287-294, 1994
168. Wudy, S A, Dorr, H G, Solleder, C, Djalali, M, Homoki, J: Profiling steroid hormones in amniotic fluid of rnidpregnancy by routine stable isotope dilution/gas chromatography-mass spectrometry: reference values and concentrations in fetuses at risk for 21-hydroxylase deficiency. Journal of Clinical Endocrinology & Metabolism 84:2724-2728, 1999
169. Pang, S, Levine, L S, Cederqvist, L L, Fuentes, M, Riccardi, V M, Holcombe, J H, Nitowsky, H M, Sachs, G, Anderson, C E, Duchon, M A, Owens, R, Merkatz, I, New, M I: Amniotic fluid concentrations of delta 5 and delta 4 steroids in fetuses with congenital adrenal hyperplasia due to 21 hydroxylase deficiency and in anencephalic fetuses. Journal of Clinical Endocrinology & Metabolism 51:223-229, 1980
170. Barbieri, R L, Saltzman, D, Torday J S, Randall R W, Frigoletto F D, Ryan K J: Elevated concentrations of the B-subunit of human chorionic gonadotropin and testosterone in the amniotic fluid of gestations of diabetic mothers. Am. J Obstet. Gynecol. 154:1039-1043, 1986
171. Longcope, C: Adrenal and gonadal androgen secretion in normal females. [Review] [94 refs]. Clinics in Endocrinology & Metabolism 15:213-228, 1986
172. Rabinovici, J, Jaffe, R B: Development and regulation of growth and differentiated function in human and subhuman primate fetal gonads. [erratum appears in Endocr Rev 1991 Feb;12(1):90.]. [Review] [276 refs]. Endocrine Reviews 11:532-557, 1990
173. Barbieri, R L, Makris, A, Randall, R W, Daniels, G, Kistner, R W, Ryan, K J: Insulin stimulates androgen accumulation in incubations of ovarian stroma obtained from women with hyperandrogenism. J. Clin.Endocrinol. Metab. 62:904-910, 1986
174. Carpenter, M W, Canick, J A, Star, J A, Shellum, C, Somers, M: A high-sensitivity assay for amniotic fluid insulin at 14-20 weeks' gestation. Obstetrics & Gynecology 94:778-782, 1999
175. Metzger, B E, Silverman, B L, Freinkel, N, Dooley, S L, Ogata, E S, Green, O C: Amniotic fluid insulin concentration as a predictor of obesity. Archives of Disease in Childhood 65:1050-1052, 1990
176. Carpenter, M W, Canick, J A, Star, J, Carr, S R, Burke, M E, Shahinian, K: Fetal hyperinsulinism at 14-20 weeks and subsequent gestational diabetes. Obstetrics & Gynecology 87:89-93, 1996
177. Kaplan, S L, Grumbach, M M: Pituitary and placental gonadotrophins and sex steroids in the human and sub-human primate fetus. [Review] [114 refs]. Clinics in Endocrinology & Metabolism 7:487-511, 1978
178. Hofman, P L, Cutfield, W S, Robinson, E M, Bergman, R N, Menon, R K, Sperling, M A, Gluckman, P D: Insulin resistance in short children with intrauterine growth retardation. Journal of Clinical Endocrinology & Metabolism 82:402-406, 1997
179. Jaquet, D, Leger, J, Chevenne, D, Czernichow, P, Levy-Marchal, C: Intrauterine growth retardation predisposes to insulin resistance but not to hyperandrogenism in young women. Journal of Clinical Endocrinology & Metabolism 84:3945-3949, 1999
180. Ibanez, L, Ong, K, Potau, N, Marcos, M V, de Zegher, F, Dunger, D: Insulin gene variable number of tandem repeat genotype and the low birth weight, precocious pubarche, and hyperinsulinism sequence. Journal of Clinical Endocrinology & Metabolism 86:5788-5793, 2001
181. Hattersley, A T, Beards, F, Ballantyne, E, Appleton, M, Harvey, R, Ellard, S: Mutations in the glucokinase gene of the fetus result in reduced birth weight. [see comments.]. Nature Genetics 19:268-270, 1998
182. Goland, R S, Jozak, S, Warren, W B, Conwell, I M, Stark, R I, Tropper, P J: Elevated levels of umbilical cord plasma corticotropin-releasing hormone in growth-retarded fetuses. Journal of Clinical Endocrinology & Metabolism 77:1174-1179, 1993
183. Yoon, B H, Romero, R, Jun, J K, Maymon, E, Gomez, R, Mazor, M, Park, J S: An increase in fetal plasma cortisol but not dehydroepiandrosterone sulfate is followed by the onset of preterm labor in patients with preterm premature rupture of the membranes. American Journal of Obstetrics & Gynecology 179:1107-1114, 1998
184. Silverman, B L, Metzger, B E, Cho, N H, Loeb, C A: Impaired glucose tolerance in adolescent offspring of diabetic mothers. Relationship to fetal hyperinsulinism. Diabetes Care 18:611-617,1995
185. Zhang, J, Bowes, W A, Jr.: Birth-weight-for-gestational-age patterns by race, sex, and parity in the United States population. Obstetrics & Gynecology 86:200-208, 1995
186. Catalano, P M, Thomas A J, Avallone, D, Amini S B: Anthropometric Estimation of Neonatal Body Composition. Am. J Obstet. Gynecol. 173:1176-1181, 1995
187. Whitelaw A: Subcutaneous Fat in Newborn Infants of Diabetic Mothers: An Indication of Quality of Diabetic Control. Lancet i: 15-18, 1977
188. Milner, R D, Hill, D J: Fetal growth control: the role of insulin and related peptides. [Review] [81 refs]. Clinical Endocrinology 21:415-433, 1984
189. Hocher, B, Slowinski, T, Bauer, C, Halle, H: The advanced fetal programming hypothesis. Nephrology Dialysis Transplantation 16:1298-1299, 2001
190. Taylor, S I, Cama, A, Accili, D, Barbetti, F, Quon, M J, de la Luz, S, Suzuki, Y, Koller, E, Levy-Toledano, R, Wertheimer, E: Mutatio ns in the insulin receptor gene. Endocr. Rev. 13:566-595, 1992
191. Bezerra R M, de Castro V, Sales T, Passini R, Marba S T M, Saad S T O, Saad M J: The Gly972Arg Polymorphism in Insulin Receptor Substrate-1 Is Associated With Decreased Birth Weight in a Population-Based Sample of Brazilian Newborns. Diabetes Care 25:550-553, 2002
192. Joshi, R, Dunaif, A: Ovarian disorders of pregnancy. Endocrinol. Metab. Clin. North Am. 24:153-169, 1995
193. Gerich, J E: The genetic basis of type 2 diabetes mellitus: impaired insulin secretion versus impaired insulin sensitivity. Endocr. Rev. 19:491-503, 1998
194. Azziz, R, Hincapie, L A, Knochenhauer, E S, Dewailly, D, Fox, L, Boots, L R: Screening for 21-hydroxylase-deficient nonclassic adrenal hyperplasia among hyperandrogenic women: a prospective study. Fertil. Steril. 72:915-925, 1999
195. Osei, K, Schuster, D P: Effects of race and ethnicity on insulin sensitivity, blood pressure, and heart rate in three ethnic populations: comparative studies in African-Americans, African immigrants (Ghanaians), and white Americans using ambulatory blood pressure monitoring. Am. J. Hypertens. 9:1157-1164, 1996
196. Modan, M, Harris, M I, Halkin, H: Evaluation of WHO and NDDG criteria for impaired glucose tolerance. Results from two national samples. Diabetes 38:1630-1635, 1989
197. Azziz, R, Dewailly, D, Owerbach, D: Clinical review 56: Nonclassic adrenal hyperplasia: current concepts. J. Clin. Endocrinol. Metab. 78:810-815, 1994
198. Legro, R S, Kunselnan, A R, Dodson, W C, Dunaif, A: Prevalence and predictors of risk for type 2 diabetes mellitus and impaired glucose tolerance in polycystic ovary syndrome: a prospective, controlled study in 254 affected women. J. Clin. Endocrinol. Metab. 84:165-169, 1999
199. Ehrmann, D A, Barnes, R B, Rosenfield, R L, Cavaghan, M K, Imperial, J: Prevalence of impaired glucose tolerance and diabetes in women with polycystic ovary syndrome. Diabetes Care 22:141-146, 1999
200. Fuleihan, G E, Testa, M, Angell, J, Porrino, N, Leboff, M: Reproducibility of DXA Absorptiometry: A Model for Bone Loss Estimates. J. Bone. Miner. Res. 10:1004-1014, 1995
201. Jensen, M D, Kanaley, J A, Reed, J E, Sheedy, P F: Measurement of abdominal and visceral fat with computed tomography and dual-energy x-ray absorptiometry. Am. J. Clin. Nutr. 61:274-278, 1995
202. Ogata, E S, Freinkel, N, Metzger, B E, Phelps, R L, Depp, R, Boehm, J J, Dooley, S L: Perinatal islet function in gestational diabetes: assessment by cord plasma C-peptide and amniotic fluid insulin. Diabetes Care 3:425-429, 1980
203. Van Assche, F A, Dallequin, M C, Holemans, K: Glucose and insulin concentration in amniotic fluid and in maternal blood in early and in late pregnancy. Diabetes Research & Clinical Practice-Supplement 10:133-135, 1990
204. Fallucca, F, Sciullo, E, Napoli, A, Cardellini, G, Maldonato, A: Amniotic fluid insulin and C peptide levels in diabetic and nondiabetic women during early pregnancy. Journal of Clinical Endocrinology & Metabolism 81:137-139, 1996
205. Carpenter, M W, Canick, J A, Hogan, J W, Shellum, C, Somers, M, Star, J A: Amniotic fluid insulin at 14-20 weeks' gestation: association with later maternal glucose intolerance and birth macrosomia. Diabetes Care 24:1259-1263, 2001
206. Dunaif, A, Scott, D, Finegood, D, Quintana, B, Whitcomb, R: The insulin-sensitizing agent troglitazone improves metabolic and reproductive abnormalities in the polycystic ovary syndrome. J. Clin. Endocrinol. Metab. 81:3299-3306, 1996
207. Orskov, C, Holst, J J: Radio-immunoassays for glucagon-like peptides 1 and 2 (GLP-1 and GLP-2). Scandinavian Journal of Clinical & Laboratory Investigation 47:165-174, 1987
208. Getty, L, Panteleon, A E, Mittelman, S D, Dea, M K, Bergman, R N: Rapid oscillations in omental lipolysis are independent of changing insulin levels in vivo. J. Clin. Invest. 106:421-430, 2000
209. Dunaif, A: Do androgens directly regulate gonadotropin secretion in the polycystic ovary syndrome? J. Clin. Endocrinol. Metab. 63:215-221, 1986
210. Anthropometric standardization reference manual. Champaign, Human Kinetic Books, 1988,

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A method to determine the presence or absence of polycystic ovary syndrome (PCOS) in an individual, comprising:
a) providing DNA or RNA from the individual; and
b) assessing the DNA or RNA for the presence or absence of the PCOS-associated allele A8(+) of a base pair polymorphism designated herein D19S884; wherein, as a result, the absence of the allele indicates a likely absence of a PCOS causative gene in the genome of the individual and the presence of the allele a likely presence of the PCOS causative gene in the genome of the individual.

2. The method of claim 1, wherein the assessing step is performed by a process that comprises subjecting the DNA or RNA to amplification using oligonucleotide primers flanking at least a portion of D19S884.

3. The method of claim 1, further comprising the step of treating said individual to prevent or ameliorate PCOS.

4. A kit for the detection of the presence or absence of a PCOS-associated allele of D19S884 comprising reagents for detecting said allele and instructions for correlating the presence or absence of said allele to a medical intervention.
